(19) United States
(12) Patent Application Publication
(10) Pub. No.: US 2026/0176673 A1
(43) Pub. Date: Jun. 25, 2026

(54) NUCLEIC ACID PROBES COMPRISING MONOMETHINE CYANINE DYES AND USES THEREOF

(71) Applicant: 10x Genomics, Inc., Pleasanton, CA (US)

(72) Inventor: Justin COSTA, Union City, CA (US)

(21) Appl. No.: 19/425,681

(22) Filed: Dec. 18, 2025

Related U.S. Application Data
(60) Provisional application No. 63/737,312, filed on Dec. 20, 2024.

(51) Int. Cl. C12Q 1/6816; C12Q 1/6809; C12Q 1/6841; C12Q 1/6876

(52) U.S. Cl. CPC C12Q 1/6816; C12Q 1/6809; C12Q 1/6841; C12Q 1/6876

(57) ABSTRACT

The present disclosure relates in some aspects to methods and systems for analyzing a biological sample comprising contacting the biological sample with a nucleic acid probe non-covalently associated with a monomethine cyanine dye or salt thereof. In some aspects, a signal associated with the monomethine cyanine dye or salt thereof is detected to determine the location of a target analyte in the biological sample.

### CROSS-REFERENCE TO RELATED APPLICATIONS

This application claims priority to and the benefit of U.S. Provisional Patent Application No. 63/737,312, filed Dec. 20, 2024, which is incorporated herein by reference in its entirety.

### FIELD

The present disclosure generally relates to methods and systems for in situ detection of analytes present in a biological sample.

### BACKGROUND

Genomic, transcriptomic, and proteomic profiling of cells and tissue samples using detectably-labeled probes and imaging can provide valuable information regarding analyte abundance and localization in situ. Thus, these in situ assays are important tools, for example, for understanding the molecular basis of cell identity, biological processes, and diseases. However, attaching or coupling detectable labels (e.g., fluorescent moieties) to these probes often requires complex, expensive, and time-consuming synthesis protocols and post-synthesis purification, thereby increasing cost and time spent performing said in situ assays. There is a need for new and improved methods for providing detectably-labeled probes for in situ assays. The present disclosure addresses these and other needs.

### SUMMARY

In some aspects, the methods and systems of the present disclosure provide means of performing an in situ assay using a nucleic acid probe comprising a detectable moiety that is not covalently attached to the nucleic acid probe, thereby enabling a simpler and more cost-effective approach for detecting an analyte at a location in a biological sample. In some aspects, the methods of the present disclosure provide a means of preparing a nucleic acid probe comprising a fluorescent moiety that does not involve covalent attachment of the fluorescent moiety to the nucleic acid probe, thereby enabling a simpler and more cost-effective approach for providing the nucleic acid probe for use in an in situ assay. In other aspects, the methods and systems of the present disclosure provide a means of improved in situ analysis through increased signal intensity associated with a nucleic acid probe used to analyze the biological sample. In some aspects, the methods and systems provided herein address issues with providing nucleic acid probes coupled to fluorescent moieties for use in detection of analytes or products thereof at location in the biological sample. In some aspects, a method disclosed herein provides a cost-effective and efficient means of detecting an analyte at a location in a biological sample using fluorescent moieties.

In some aspects, provided herein is a method, including: (a) contacting a biological sample including a target nucleic acid, or an extension or amplification product thereof with a nucleic acid probe and a monomethine cyanine dye or a salt thereof, wherein the nucleic acid probe is non-covalently associated with the monomethine cyanine dye or the salt thereof, thereby hybridizing the nucleic acid probe to the target nucleic acid or the extension or amplification product thereof at a location in the biological sample; and (b) detecting, at the location, a signal associated with the monomethine cyanine dye or the salt thereof.

In some aspects, provided herein is a method, wherein the monomethine cyanine dye or the salt thereof is selected from the group consisting of a thioalkyl derivative, 2-iminobenzo-thiazoline, a sulfobetaine salt of N-alkyl heterocycle, 2-chloroquinoline, 4-chloroquinoline, 3-phenyl-2H-1,4-benzothiazine, indole-3-carboxaldehyde, methyl-pyridinium iodide, thiazole orange or a derivative thereof, oxazole yellow or a derivative thereof, and oxazole blue or a derivative thereof.

In some aspects, provided herein is a method, wherein the nucleic acid probe is in a single-stranded form prior to the hybridizing.

In some aspects, provided herein is a method, wherein the monomethine cyanine dye is a monomeric monomethine cyanine dye.

In some aspects, provided herein is a method, wherein the monomeric monomethine cyanine dye has the structure:

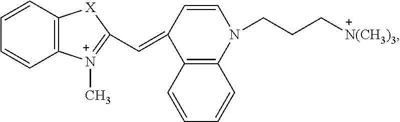

wherein X is O or S.

In some aspects, provided herein is a method, wherein the monomeric monomethine cyanine dye has the structure:

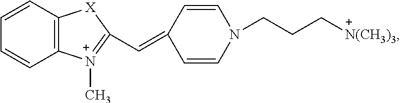

wherein X is O or S.

In some aspects, provided herein is a method, wherein the monomeric monomethine cyanine dye has the structure:

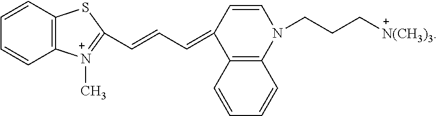

In some aspects, provided herein is a method, wherein the nucleic acid probe is in a partially double-stranded form prior to the hybridizing.

In some aspects, provided herein is a method, wherein the partially double-stranded form includes a hairpin form.

In some aspects, provided herein is a method, wherein the monomethine cyanine dye is a dimeric monomethine cyanine dye.

In some aspects, provided herein is a method, wherein the dimeric monomethine cyanine dye has the structure:

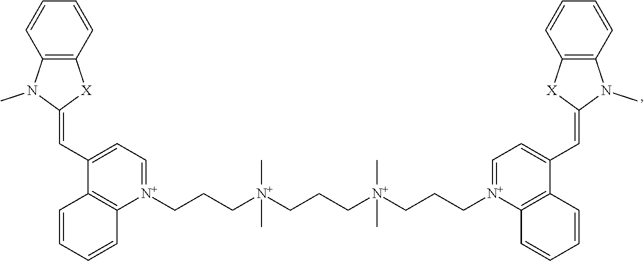

wherein X is S or O.

In some aspects, provided herein is a method, wherein the dimeric monomethine cyanine dye has the structure:

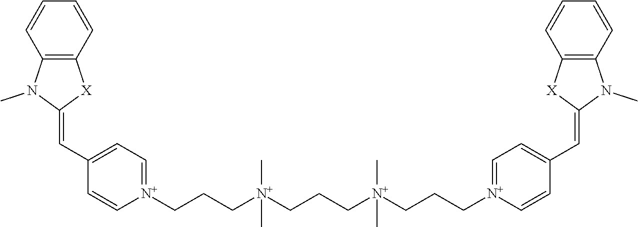

wherein X is S or O.

In some aspects, provided herein is a method, wherein the dimeric monomethine cyanine dye has the structure:

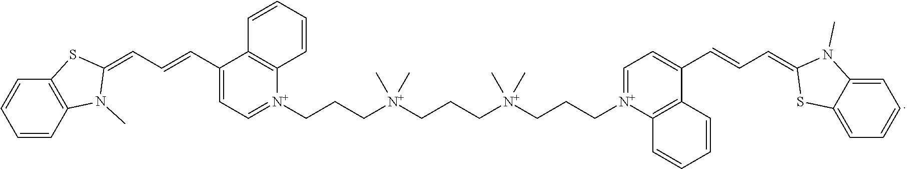 [SMILES: CN1/C(=C/C=C/c2cc[n+](CCC[N+](C)(C)CCC[N+](C)(C)CCC[n+]3ccc(/C=C/C=C4/Sc5ccccc5N4C)c4ccccc43)c3ccccc23)Sc2ccccc21]

In some aspects, provided herein is a method, further including, prior to the contacting, incubating the nucleic acid probe in a solution including the monomethine cyanine dye or the salt thereof, thereby non-covalently associating the nucleic acid probe with the monomethine cyanine dye or the salt thereof.

In some aspects, provided herein is a method, wherein the contacting is for a duration of about 30 seconds to about 2 minutes.

In some aspects, provided herein is a method, wherein the contacting includes hybridizing the nucleic acid probe to a rolling circle amplification product (RCP) associated with a target nucleic acid at the location in the biological sample.

In some aspects, provided herein is a method, wherein the RCP is generated from a circular template at the location in the biological sample.

In some aspects, provided herein is a method, further including contacting the biological sample with a circularizable probe or probe set that directly or indirectly binds to the target nucleic acid at the location in the biological sample, and circularizing the circularizable probe or probe set to generate the circular template.

In some aspects, provided herein is a method, wherein the detecting includes detecting a fluorescence signal of the monomethine cyanine dye or the salt thereof using fluorescence-based microscopy.

In some aspects, provided herein is a method, including: (a) contacting a biological sample including an amplicon of a target nucleic acid molecule with (i) a nucleic acid probe including a single stranded region and a double-stranded region, and (ii) a double-intercalating monomethine cyanine dye or a salt thereof that is non-covalently associated with the double-stranded region, thereby hybridizing the nucleic acid probe to the amplicon of the target nucleic acid molecule at a location in the biological sample; and (b) detecting, at the location, a signal associated with the monomethine cyanine dye or the salt thereof.

In some aspects, provided herein is a system for detecting a target nucleic acid, the system including: (a) a biological sample including a target nucleic acid or an extension or amplification product thereof; and (b) a nucleic acid probe non-covalently associated with a monomethine cyanine dye or a salt thereof, wherein the nucleic acid probe is hybridized to the target nucleic acid or the extension or amplification product thereof.

In some aspects, provided herein is a kit including: (a) a circularizable probe or probe set that is complementary to a target nucleic acid of a biological sample; and (b) a nucleic acid probe non-covalently associated with a monomethine cyanine dye or a salt thereof, wherein the nucleic acid probe includes a sequence complementary to an extension or amplification product of the target nucleic acid.

In some aspects, provided herein is a method comprising a) contacting a biological sample with a nucleic acid probe, wherein the nucleic acid probe is non-covalently associated with a monomethine cyanine dye or a salt thereof, wherein the contacting results in the nucleic acid probe binding directly or indirectly to a target nucleic acid at a location in the biological sample; and b) detecting a signal associated with the monomethine cyanine dye or the salt thereof.

In some aspects, provided herein is a method comprising: a) contacting a biological sample with a nucleic acid probe, wherein the nucleic acid probe is non-covalently associated with a monomethine cyanine dye or a salt thereof, wherein the contacting results in the nucleic acid probe binding to a target nucleic acid or an extension/amplification product thereof at a location in the biological sample; and b) detecting a signal associated with the monomethine cyanine dye or the salt thereof.

In some embodiments, the monomethine cyanine dye or the salt thereof comprises and/or is derived from a thioalkyl derivative, 2-iminobenzo-thiazoline, a sulfobetaine salt of N-alkyl heterocycle, 2-chloroquinoline, 4-chloroquinoline, 3-phenyl-2H-1,4-benzothiazine, indole-3-carboxaldehyde, methyl-pyridinium iodide, thiazole orange or a derivative thereof, oxazole yellow or a derivative thereof, or oxazole blue or a derivative thereof. In some embodiments, the monomethine cyanine dye or the salt thereof is derived from thiazole orange, oxazole yellow, or oxazole blue. In some embodiments, the monomethine cyanine dye or the salt thereof is monomeric. In some embodiments, the monomethine cyanine dye or the salt thereof is dimeric.

In some aspects, provided herein is a method comprising: a) providing a nucleic acid probe that is non-covalently associated with a monomethine cyanine dye or salt thereof, b) contacting a biological sample with the nucleic acid probe, wherein the contacting results in the nucleic acid probe binding directly or indirectly to a target nucleic acid at a location in the biological sample; and c) detecting a signal associated with the monomethine cyanine dye or the salt thereof.

In some aspects, provided herein is a method comprising: a) providing a nucleic acid probe that is non-covalently associated with a monomethine cyanine dye or salt thereof, b) contacting a biological sample with the nucleic acid probe, wherein the contacting results in the nucleic acid probe binding to a target nucleic acid or extension/amplification product thereof at a location in the biological sample; and c) detecting a signal associated with the monomethine cyanine dye or the salt thereof.

In some embodiments, the nucleic acid probe is provided in a single-stranded form.

In some embodiments, the monomethine cyanine dye is a monomeric monomethine cyanine dye. In some embodiments, the monomeric monomethine cyanine dye has the structure:

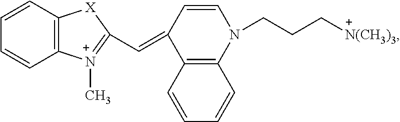

wherein X is O or S. In some embodiments, the monomeric monomethine cyanine dye has the structure:

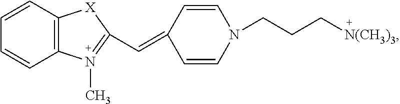

wherein X is O or S. In some embodiments, the monomeric monomethine cyanine dye has the structure:

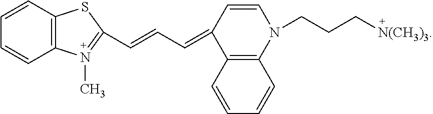

In some embodiments, the nucleic acid probe is provided in partially double-stranded form. In some embodiments, the partially double-stranded form comprises a hairpin form.

In some embodiments, the monomethine cyanine dye is a dimeric monomethine cyanine dye. In some embodiments, the dimeric monomethine cyanine dye has a structure of formula:

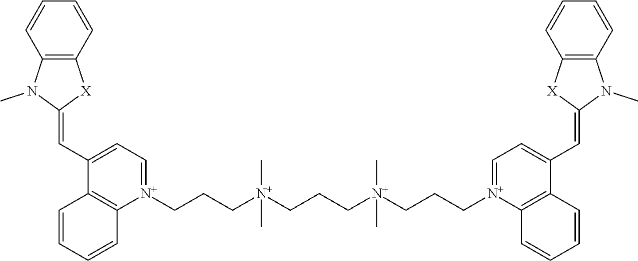

wherein X is S or O. In some embodiments, the dimeric monomethine cyanine dye has a structure of formula:

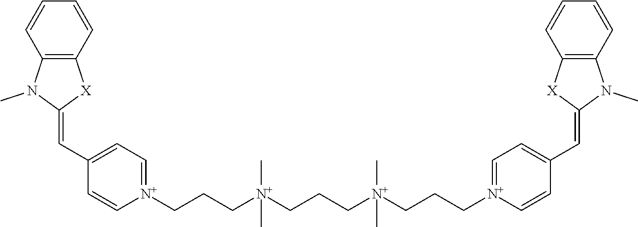

wherein X is S or O. In some embodiments, the dimeric monomethine cyanine dye has the structure:

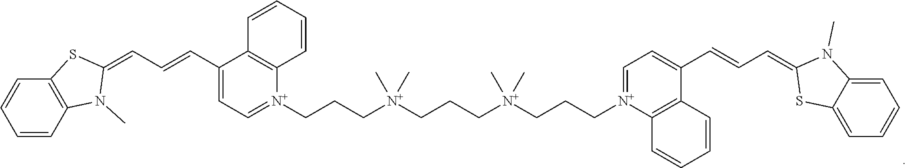 [SMILES: CN1/C(=C/C=C/c2cc[n+](CCC[N+](C)(C)CCC[N+](C)(C)CCC[n+]3ccc(/C=C/C=C4/Sc5ccccc5N4C)c4ccccc43)c3ccccc23)Sc2ccccc21]

In some embodiments, the nucleic acid probe is RNA.

In some embodiments, the nucleic acid probe is DNA.

In some embodiments, providing the nucleic acid probe with the monomethine cyanine dye or the salt thereof comprises incubating a nucleic acid molecule in a solution comprising the monomethine cyanine dye or the salt thereof.

In some embodiments, the contacting is for a duration of about 30 seconds to about 2 minutes.

In some embodiments, the solution is an aqueous solution.

In some embodiments, the solution is an organic solution.

In some embodiments, the nucleic acid probe hybridizes to a rolling circle amplification product (RCP) associated with the target nucleic acid at the location in the biological sample. In some embodiments, the RCP is generated from a circular template at the location in the biological sample. In some embodiments, the method comprises contacting the biological sample with the circular template. In some embodiments, the circular template is a circular probe that directly or indirectly binds to the target nucleic acid at the location in the biological sample. In some embodiments, the method comprises generating the circular template at the location in the biological sample. In some embodiments, the method comprises reverse transcribing an RNA in the biological sample to generate a molecule comprising a cDNA of the RNA and circularizing the molecule comprising the cDNA to generate the circular template. In some embodiments, the method comprises contacting the biological sample with a circularizable probe or probe set that directly or indirectly binds to the target nucleic acid at the location in the biological sample, and circularizing the circularizable probe or probe set to generate the circular template. In some embodiments, the circularizable probe or probe set hybridizes to the target nucleic acid, and the circularizable probe or probe set is circularized using the target nucleic acid as a template. In some embodiments, hybridization of the circularizable probe or probe set to the target nucleic acid forms a gap between the ends of the circularizable probe or probe set, and wherein generating the circular template comprises filling the gap between the ends of the circularizable probe or probe set.

In some embodiments, the circular template does not comprise a barcode sequence assigned to associate with the target nucleic acid or a sequence thereof.

In some embodiments, the circular template comprises a barcode sequence assigned to associate with the target nucleic acid or a sequence thereof.

In some embodiments, the target nucleic acid is a cellular nucleic acid in the biological sample. In some embodiments, the cellular nucleic acid is genomic DNA. In some embodiments, the cellular nucleic acid is RNA. In some embodiments, the RNA is mRNA, miRNA, lnRNA, or rRNA. In some embodiments, the RNA is mRNA.

In some embodiments, the target nucleic acid is a reporter oligonucleotide comprised on a labeling agent, wherein the labeling agent is bound to an analyte in the biological sample. In some embodiments, the analyte is a nucleic acid analyte. In some embodiments, the analyte is a polypeptide.

In some embodiments, the signal associated with the monomethine cyanine dye or the salt thereof is a fluorescence signal. In some embodiments, the method comprises detecting the fluorescence signal using fluorescence-based microscopy.

In some embodiments, the biological sample is a fresh tissue sample, a frozen tissue sample, or a fixed tissue sample.

In some embodiments, the biological sample is a fresh frozen tissue section or a formalin-fixed paraffin-embedded tissue section.

In some embodiments, the biological sample is derived from a human. In some embodiments, the biological sample is derived from a human with a disease or condition.

In some embodiments, the biological sample is derived from a non-human mammal.

In some aspects, provided herein is a method comprising: a) contacting a biological sample with a nucleic acid probe, wherein the nucleic acid probe is non-covalently associated with a monomethine cyanine dye or a salt thereof, wherein the nucleic acid probe hybridizes to a region of a rolling circle amplification product (RCP) at a location in the biological sample, wherein the RCP is associated with a target nucleic acid in the biological sample; and b) detecting a signal associated with the monomethine cyanine dye or salt thereof, thereby detecting the target nucleic acid in the biological sample.

In some embodiments, the monomethine cyanine dye or the salt thereof comprises and/or is derived from a thioalkyl derivate, 2-iminobenzo-thiazoline, a sulfobetaine salt of N-alkyl heterocycle, 2-chloroquinoline, 4-chloroquinoline, 3-phenyl-2H-1,4-benzothiazine, indole-3-carboxaldehyde, methyl-pyridinium iodide, thiazole orange or a derivative thereof, oxazole yellow or a derivative thereof, or oxazole blue or a derivative thereof. In some embodiments, the monomethine cyanine dye or the salt thereof is monomeric. In some embodiments, the monomethine cyanine dye or the salt thereof is dimeric.

In some aspects, provided herein is a method comprising: a) providing a nucleic acid probe non-covalently associated with a monomethine cyanine dye; b) contacting a biological sample with the nucleic acid probe, wherein the nucleic acid probe hybridizes to a region of a rolling circle amplification product (RCP) at a location in the biological sample, wherein the RCP is associated with a target nucleic acid in the biological sample; and c) detecting a signal associated with the monomethine cyanine dye or salt thereof, thereby detecting the target nucleic acid in the biological sample.

In some embodiments, the nucleic acid probe is provided in a single-stranded form.

In some embodiments, the monomethine cyanine dye is a monomeric monomethine cyanine dye.

In some embodiments, the nucleic acid probe is provided in a partially double-stranded form.

In some embodiments, the monomethine cyanine dye is a dimeric monomethine cyanine dye.

In some aspects, provided herein is a method comprising: a) contacting a biological sample with a nucleic acid probe, wherein the nucleic acid probe comprises a hairpin form, wherein the nucleic acid probe is non-covalently associated with a double-intercalating monomethine cyanine dye or a salt thereof, wherein the nucleic acid probe hybridizes to a region of a rolling circle amplification product (RCP), wherein the RCP is associated with a target nucleic acid at a location in the biological sample; and b) detecting a signal associated with the monomethine cyanine dye or salt thereof.

In some aspects, provided herein is a system for detecting a target nucleic acid, the system comprising: a) a biological sample comprising a target nucleic acid or an extension or amplification product thereof; and b) a nucleic acid probe non-covalently associated with a monomethine cyanine dye or a salt thereof, wherein the nucleic acid probe is hybridized to the target nucleic acid or the extension or amplification product thereof.

In some embodiments, the nucleic acid probe is hybridized to the extension or amplification product thereof.

In some embodiments, the extension or amplification product is a rolling circle amplification product.

In some embodiments, the biological sample is on a slide or in a well.

In some embodiments, the system further comprises an imaging device, and wherein the slide or the well is on the imaging device.

In some aspects, provided herein is a kit comprising: (a) a circularizable probe or probe set that is complementary to a target nucleic acid of a biological sample; and (b) a nucleic acid probe non-covalently associated with a monomethine cyanine dye or a salt thereof, wherein the nucleic acid probe comprises a sequence complementary to an extension or amplification product of the target nucleic acid.

In some embodiments, the kit further comprises a ligase for forming a circular template from the circularizable probe or probe set.

In some embodiments, the kit further comprises a polymerase suitable for a gapfill reaction.

In some embodiments, the kit further comprises a mixture of free nucleotides including the four canonical bases: adenine, thymine, guanine, and cytosine.

In some embodiments, the kit further comprises a polymerase suitable for rolling circle amplification.

In some embodiments, the circularizable probe comprises a primer binding sequence, and the kit further comprising a primer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings illustrate certain features and advantages of this disclosure. These embodiments are not intended to limit the scope of the appended claims in any manner.

FIGS. 1A-1B show an example of preparing and using a nucleic acid probe non-covalently associated with a monomethine cyanine dye for detection of a target nucleic acid or a probe or product thereof. FIG. 1A shows general schematic to forming a nucleic acid probe. FIG. 1B shows detection of a rolling circle amplification product (RCP) using a nucleic acid probe.

FIG. 2 shows example monomethine cyanine dyes (MCDs) To-Pro3, To-Prol, Yo-Prol, and Po-Prol and corresponding structures and physical parameters (e.g., excitation/emission wavelengths and molecular weight) for use in preparing the nucleic acid probe.

FIGS. 3A-3D show an example multiplex assay for detecting a probe product (e.g., an RCP) in a biological sample. FIG. 3A shows the multiplex assay approach wherein a single RCP is contacted with a nucleic acid probe and an anchor probe. FIG. 3B shows detection of the signal associated with the anchor probe using fluorescence microscopy. FIG. 3C shows detection of the signal associated with the anchor probe and the signal associated with the nucleic acid probe using fluorescence microscopy. FIG. 3D shows detection of the RCP using the anchor probe 647N (red) channel only following stripping of the probes from the RCP and rehybridization of the anchor probe only.

FIGS. 4A-4B demonstrate detection of a probe or probe product (e.g., an RCP) in a biological sample using a nucleic acid probe provided in a partially double-stranded form (e.g., in a hairpin form). FIG. 4A shows labeling of a hairpin-form nucleic acid probe with a monomethine cyanine dye, while FIG. 4B shows detection of an RCP using the hairpin-form nucleic acid probe.

FIG. 5 is an example workflow of analysis of a biological sample (e.g., a cell or tissue sample) using rolling circle amplification (RCA).

FIG. 6 is an example workflow of analysis of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

The ability to locate target analytes (e.g., target nucleic acids) of interest within a biological sample (e.g., a tissue or a cell) holds the potential to propel our understanding of biology by revealing spatial expression patterns correlated to or causally associated with normal and disease states of interest. One such approach for spatial analysis of target analytes (e.g., target nucleic acids) comprises in situ hybridization of detectably-labeled probes comprising detectable labels (e.g., fluorophores and/or fluorescent moieties) directly or indirectly to the target analytes of interest, followed by detection of signals associated with the detectable labels. For example, a target nucleic acid may be contacted with a circularizable probe comprising a sequence that identifies the target nucleic acid, followed by circularization of the probe using the target nucleic acid as a template, and amplification using rolling circle amplification to generate a rolling circle amplification product (RCP), wherein the RCP comprises multiple copies of the complement of the sequence that identifies the target nucleic acid. Binding of detectably-labeled probes to the amplified copies of the complement of the sequence of interest provides an amplified signal, which can then be detected to identify the location of the target nucleic acid at a location in a biological sample.

One limitation to this approach is the production of the detectably-labeled probes. Generating detectably-labeled probes typically requires conjugation (e.g., covalent attachment) of the detectable label (e.g., the fluorophore or the fluorescent moiety) to a nucleic acid molecule. This process can be costly and time consuming, as it requires complex attachment chemistry and downstream purification methods to prepare the detectably-labeled probes. Another concern is low fluorescent output per detectably-labeled probe, as there may be a limit to the number of fluorophores that can be conjugated to a given nucleic acid molecule. Yet another limitation to this approach is the ability of detectably-labeled probes to bind/hybridize complementary nucleic acid sequences (e.g., target sequences comprised on a probe product) once conjugated to the detectable label. Without being bound by theory, detectable labels (e.g., fluorophores) can be relatively large molecules, and detectably-labeled probes comprising such bulky label attachments may exhibit decreased efficiency of binding to targets because of steric hindrance and/or interference by the attachments with Watson-Crick base-pairing. Given these limitations, new solutions are needed to provide probes that comprise detectable labels and are compatible with in situ experimental approaches while not being too costly or inefficient to produce.

Monomethine cyanine dyes are fluorescent dyes that exhibit a concomitant increase in fluorescent emission and/or fluorescent growth following non-covalent association with various biomacromolecules such as nucleic acids (e.g., DNA or RNA). In general, cyanine dyes are fluorescent dyes that comprise a cyanine chromophore, which is a polymethine chain with conjugated —C═C— bonds that typically connect N-containing end heterocycles and has an odd number of carbon atoms. The spectral range of emission and absorption of each cyanine dye is primarily influenced by the length of the polymethine chain. Monomethine cyanine dyes are a class of cyanine dyes comprising only one methine group within its polymethine chain (i.e., comprises a monomethine chain) linking terminal heterocyclic groups. More information on the nature of cyanine dyes and monomethine cyanine dyes in particular can be found, for example, in Pronkin and Tatikolov, “Fluorescent Probes for Biomacromolecules Based on Monomethine Cyanine Dyes”, Chemosensors, (2023), 11:280, 1-35, the content of which is herein incorporated by reference in its entirety.

Monomethine cyanine dyes have been employed as fluorescent probes to directly detect nucleic acids, given their ability to non-covalently bind these molecules. For example, SYBR Green I is an asymmetrical cyanine dye that intercalates dsDNA for quantification of these molecules in techniques such as gel electrophoresis and real-time quantitative qPCR. Based on these principles, monomethine cyanine dyes may be employed to generate probes comprising detectable labels (e.g., nucleic acid probes) based off the same or similar non-covalent interaction. Without being bound by theory, preparation of nucleic acid probes comprising non-covalent association with monomethine cyanine dyes (rather than fluorophore-conjugated detectably-labeled probes) may address at least one or more issues with providing probes for in situ analysis of target analytes in biological samples.

Provided herein are methods directed to the use of nucleic acid probes comprising non-covalent association to monomethine cyanine dyes or salts thereof for analysis of target analytes in situ. In some embodiments, the nucleic acid probe comprises a nucleic acid molecule associated with a monomeric cyanine dye or derivative thereof or salt thereof. In some embodiments, the nucleic acid probe is formed and/or produced by incubating a mixture comprising a nucleic acid molecule and a monomethine cyanine dye or a salt thereof in vitro. In some embodiments, the incubation is sufficiently long to facilitate near complete or complete association between the nucleic acid molecule and the monomethine cyanine dye. Without being bound by theory, non-covalent association of the nucleic acid molecule to the monomethine cyanine dye or salt thereof may be achieved through electrostatic binding of the dye to the nucleic acid molecule (e.g., through π-stacking with the quinoline ring between two dA bases) and/or intercalation of the dye with the nucleic acid molecule (e.g., intercalation of the dye with the minor groove of dsDNA). In some embodiments, the method comprises providing the nucleic acid probe formed/produced through the mixing to an in situ assay.

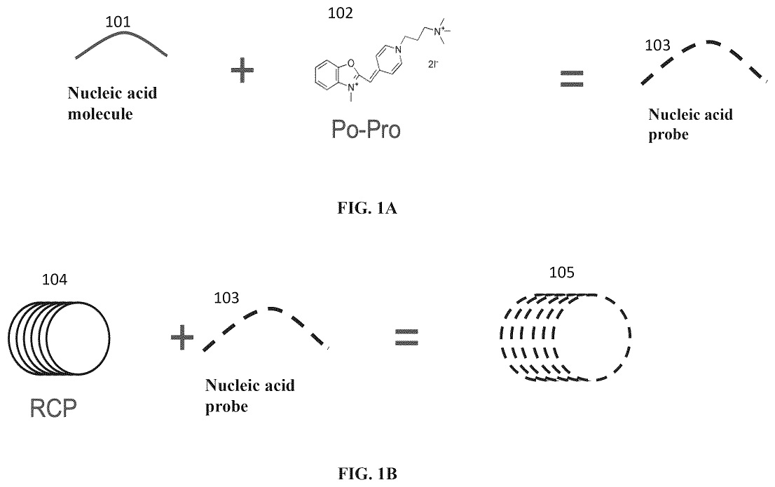

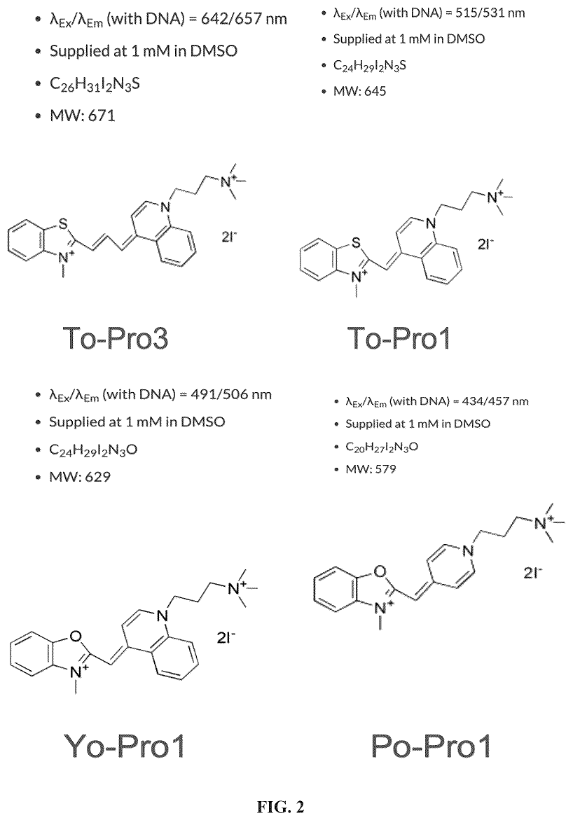

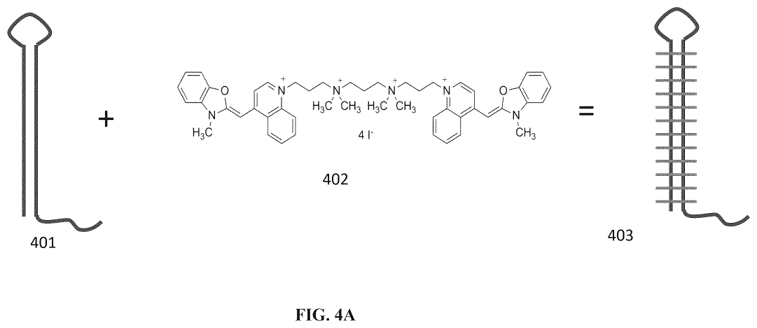

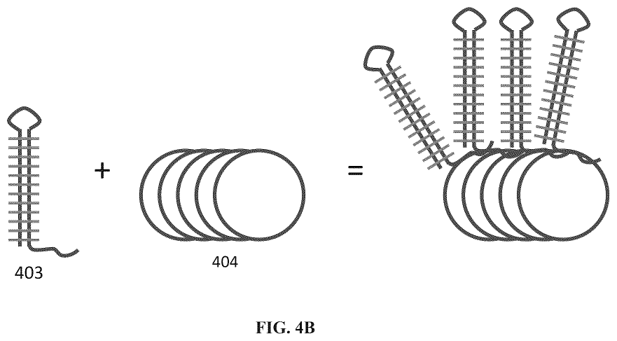

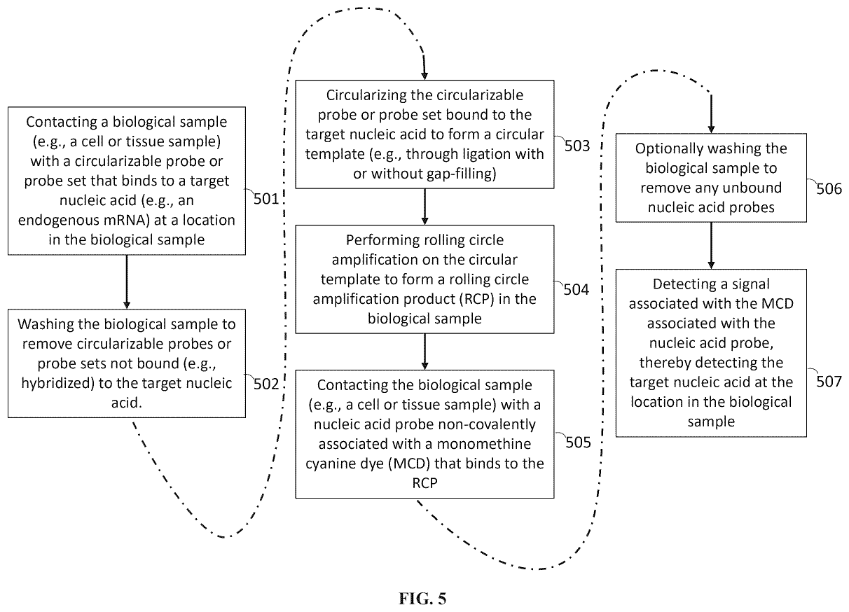

In some embodiments, the methods comprise contacting a biological sample with the nucleic acid probe. In some embodiments, the nucleic acid probe is sufficiently complementary to or fully complementary to a target sequence. In some embodiments, one or more portions of the nucleic acid probe is sufficiently complementary to or fully complementary to a target sequence. In some embodiments, a target analyte comprises the target sequence. Target analytes include, but are not limited to, those described in Section III.B. In some embodiments, the target analyte is a target nucleic acid. In some embodiments, the target nucleic acid comprises the target sequence. In some embodiments, a product of a target nucleic acid comprises the target sequence. In some embodiments, a probe or probe set associated with a target nucleic acid comprises the target sequence. In some embodiments, a product of a probe or probe set associated with a target nucleic acid comprises the target sequence. In some embodiments, a labeling agent that binds to the target analyte comprises the target sequence. In some embodiments, the labeling agent comprises a reporter oligonucleotide that comprises the target sequence. Example probes and probes sets, and generation of products thereof, are described in Section II.B.(ii). In some embodiments, the contacting results in hybridization of the nucleic acid probe to the target sequence in the biological sample. In some embodiments, the hybridization of the nucleic acid probe to the target sequence results in direct or indirect association of the nucleic acid probe to the target nucleic acid at a location in the biological sample. Non-limiting example schematics of the association/interaction between a nucleic acid probe and a target sequence (e.g., a target sequence comprised on a probe product such as an RCP) are shown in FIG. 1B and FIG. 4B.

In some embodiments, after the nucleic acid probe is directly or indirectly associated/bound to the target analyte (e.g., target nucleic acid), the method comprises detecting a signal associated with the monomethine cyanine dye or the salt thereof. In some embodiments, the signal is a fluorescent signal. In some embodiments, the fluorescent signal has a known emission wavelength. In some embodiments, detecting the signal comprises imaging the biological sample. In some embodiments, the imaging comprises fluorescence microscopy. Example signal detection and/or imaging approaches, including example fluorescence microscopy approaches, as described in Section II.C.(iii) can be applied for the detection of the signal associated with the monomethine cyanine dye or the salt thereof. In some embodiments, detecting the signal associated with the monomethine cyanine dye or the salt thereof identifies the location of the target nucleic acid within the biological sample.

In some embodiments, the methods and systems disclosed herein provide one or more advantages compared to conventional approaches for in situ analysis comprising use of detectably-labeled probes. In one aspect, the methods and systems disclosed herein are used to increase the brightness and/or fluorescence intensity of nucleic acid probes used for detection of target analytes at a location in a biological sample. Without being bound by theory, this may be achieved by increasing the abundance of fluorescent moieties associated with the nucleic acid probe through non-covalent association of monomethine cyanine dyes rather than covalent attachment of other fluorophores. In another aspect, the methods and systems disclosed herein decrease the cost and time for producing/providing the nucleic acid probes, for example by avoiding costly and time-consuming conjugation steps and post-conjugation purification steps. In some embodiments, the methods and systems disclosed herein improve the association and/or hybridization of nucleic acid probes to complementary target nucleic acid sequences by preventing the occurrence of steric hindrance between the nucleic acid probe and target sequence/target analyte, and/or avoiding bulk conjugated moieties (e.g., fluorescent moieties or fluorophores) that interfere with Watson-Crick base-pairing.

In some aspects, the present application provides various designs for nucleic acid probes associated with monomethine cyanine dyes or salts thereof.

Additional aspects of the methods, systems, compositions, and kits disclosed herein are described in the sections below.

### II. Methods and Systems Comprising Nucleic Acid Probes

In some aspects, provided herein are methods for analyzing a biological sample comprising the use of nucleic acid probes. In some aspects, provided herein are systems for analyzing a biological sample comprising nucleic acid probes. In some embodiments, the nucleic acid probe is non-covalently associated with a monomethine cyanine dye or a salt thereof. In some embodiments, the nucleic acid probe is provided in a single-stranded form. In some embodiments, the nucleic acid probe is provided in a partially double-stranded form (e.g., a hairpin/stem-loop form). In some embodiments, the nucleic acid probe directly or indirectly associates with a target analyte (e.g., a target nucleic acid) at a location in the biological sample. In some embodiments, a signal associated with the monomethine cyanine dye or salt thereof is detected. In some embodiments, the detection comprises imaging the biological sample. In some embodiments, the imaging is fluorescence imaging. In some embodiments, detecting the signal associated with the monomethine cyanine dye or the salt thereof thereby identifies the location of the target analyte (e.g., the target nucleic acid) within the biological sample.

In some embodiments, provided herein are methods comprising: a) contacting a biological sample with a nucleic acid probe, wherein the nucleic acid probe is non-covalently associated with a monomethine cyanine dye or a salt thereof, wherein the contacting results in the nucleic acid probe binding directly or indirectly to a target nucleic acid at a location in the biological sample; and b) detecting a signal associated with the monomethine cyanine dye or the salt thereof. In some embodiments, the nucleic acid probe is provided in a single-stranded form. In some embodiments, the nucleic acid probe is provided in partially double-stranded form. In some embodiments, the nucleic acid probe comprises a hairpin/stem-loop form. In some embodiments, the monomethine cyanine dye is monomeric. In some embodiments, the monomethine cyanine dye is dimeric. In some embodiments, the contacting in a) comprises hybridization of the nucleic acid probe to a target sequence comprised in or associated with the target nucleic acid at the location in the biological sample. In some embodiments, the nucleic acid probe hybridizes to the target nucleic acid. In some embodiments, the nucleic acid probe hybridizes to a product of the target nucleic acid. In some embodiments, the nucleic acid probe hybridizes to a probe or probe set associated with the target nucleic acid. In some embodiments, the nucleic acid probe hybridizes to a product of a probe or probe set associated with the target nucleic acid

In some embodiments, provided herein are methods comprising: a) providing a nucleic acid probe that is non-covalently associated with a monomethine cyanine dye or salt thereof, b) contacting a biological sample with the nucleic acid probe, wherein the contacting results in the nucleic acid probe annealing binding directly or indirectly to a target nucleic acid at a location in the biological sample; and c) detecting a signal associated with the monomethine cyanine dye or the salt thereof. In some embodiments, the nucleic acid probe is provided in a single-stranded form. In some embodiments, the nucleic acid probe is provided in partially double-stranded form. In some embodiments, the nucleic acid probe comprises a hairpin/stem-loop form. In some embodiments, the monomethine cyanine dye is monomeric. In some embodiments, the providing in a) comprises generating a nucleic acid probe by mixing a nucleic acid molecule with a monomethine cyanine dye or salt thereof in a solution. In some embodiments, the monomethine cyanine dye is dimeric. In some embodiments, the contacting in b) comprises hybridization of the nucleic acid probe to a target sequence comprised in or associated with the target nucleic acid at the location in the biological sample. In some embodiments, the nucleic acid probe hybridizes to the target nucleic acid. In some embodiments, the nucleic acid probe hybridizes to a product of the target nucleic acid. In some embodiments, the nucleic acid probe hybridizes to a probe or probe set associated with the target nucleic acid. In some embodiments, the nucleic acid probe hybridizes to a product of a probe or probe set associated with the target nucleic acid.

In some embodiments, provided herein are methods comprising: a) providing a nucleic acid probe that is non-covalently associated with a monomethine cyanine dye or salt thereof, wherein the nucleic acid probe is provided in single-stranded form, b) contacting a biological sample with the nucleic acid probe, wherein the contacting results in the nucleic acid probe annealing binding directly or indirectly to a target nucleic acid at a location in the biological sample; and c) detecting a signal associated with the monomethine cyanine dye or the salt thereof. In some embodiments, the monomethine cyanine dye is monomeric. In some embodiments, the providing in a) comprises generating a nucleic acid probe by mixing a nucleic acid molecule with a monomethine cyanine dye or salt thereof in a solution. In some embodiments, the monomethine cyanine dye is dimeric. In some embodiments, the contacting in b) comprises hybridization of the nucleic acid probe to a target sequence comprised in or associated with the target nucleic acid at the location in the biological sample. In some embodiments, the nucleic acid probe hybridizes to the target nucleic acid. In some embodiments, the nucleic acid probe hybridizes to a product of the target nucleic acid. In some embodiments, the nucleic acid probe hybridizes to a probe or probe set associated with the target nucleic acid. In some embodiments, the nucleic acid probe hybridizes to a product of a probe or probe set associated with the target nucleic acid.

In some embodiments, provided herein are methods comprising: a) providing a nucleic acid probe that is non-covalently associated with a monomethine cyanine dye or salt thereof, wherein the nucleic acid probe is provided in partially double-stranded form; b) contacting a biological sample with the nucleic acid probe, wherein the contacting results in the nucleic acid probe annealing binding directly or indirectly to a target nucleic acid at a location in the biological sample; and c) detecting a signal associated with the monomethine cyanine dye or the salt thereof. In some embodiments, the nucleic acid probe comprises a hairpin/stem-loop form. In some embodiments, the providing in a) comprises generating a nucleic acid probe by mixing a nucleic acid molecule with a monomethine cyanine dye or salt thereof in a solution. In some embodiments, the monomethine cyanine dye is dimeric. In some embodiments, the monomethine cyanine dye is a homodimer. In some embodiments, the contacting in b) comprises hybridization of the nucleic acid probe to a target sequence comprised in or associated with the target nucleic acid at the location in the biological sample. In some embodiments, the nucleic acid probe hybridizes to the target nucleic acid. In some embodiments, the nucleic acid probe hybridizes to a product of the target nucleic acid. In some embodiments, the nucleic acid probe hybridizes to a probe or probe set associated with the target nucleic acid. In some embodiments, the nucleic acid probe hybridizes to a product of a probe or probe set associated with the target nucleic acid.

In some embodiments, provided herein are methods comprising a) providing a nucleic acid probe non-covalently associated with a monomethine cyanine dye; b) contacting a biological sample with the nucleic acid probe, wherein the nucleic acid probe hybridizes to a region of a rolling circle amplification product (RCP) at a location in the biological sample, wherein the RCP is associated with a target nucleic acid in the biological sample; and c) detecting a signal associated with the monomethine cyanine dye or salt thereof, thereby detecting the target nucleic acid in the biological sample. In some embodiments, the nucleic acid probe is provided in a single-stranded form. In some embodiments, the nucleic acid probe is provided in partially double-stranded form. In some embodiments, the nucleic acid probe comprises a hairpin/stem-loop form. In some embodiments, the monomethine cyanine dye is monomeric. In some embodiments, the providing in a) comprises generating a nucleic acid probe by mixing a nucleic acid molecule with a monomethine cyanine dye or salt thereof in a solution. In some embodiments, the monomethine cyanine dye is dimeric. In some embodiments, the contacting in b) comprises hybridization of the nucleic acid probe to a target sequence comprised in or associated with the RCP at the location in the biological sample. In some embodiments, the RCP is generated from a circular template at the location in the biological sample. In some embodiments, the circular template is a circular probe that directly or indirectly binds to the target nucleic acid at the location in the biological sample. In some embodiments, the circular template is generated at the location in the biological sample. In some embodiments, the target nucleic acid is an RNA, and the circular template is generated by transcribing the RNA in the biological sample generate a molecule comprising a cDNA of the RNA and circularizing the molecule comprising the cDNA. In some embodiments, the circular template is generated by contacting the biological sample with a circularizable probe or probe set that directly or indirectly binds to the target nucleic acid at the location in the biological sample, and circularizing the circularizable probe or probe set. In some embodiments, provided herein are methods comprising: (a) contacting a biological sample comprising an amplicon of a target nucleic acid molecule with (i) a nucleic acid probe comprising a single stranded region and a double-stranded region, and (ii) a double-intercalating monomethine cyanine dye, or a salt thereof, that is non-covalently associated with the double-stranded region, thereby hybridizing the nucleic acid probe to the amplicon of the target nucleic acid molecule at a location in the biological sample; and (b) detecting, at the location, a signal associated with the monomethine cyanine dye, or a salt thereof. In some embodiments, the double-intercalating monomethine cyanine dye is dimeric. In some embodiments, the double-intercalating monomethine cyanine dye is a homodimer. In some other embodiments, provided herein are methods comprising: a) contacting a biological sample with a nucleic acid probe, wherein the nucleic acid probe comprises a hairpin form, wherein the nucleic acid probe is non-covalently associated with a double-intercalating monomethine cyanine dye or a salt thereof, wherein the nucleic acid probe hybridizes to a region of a rolling circle amplification product (RCP), wherein the RCP is associated with a target nucleic acid at a location in the biological sample; and b) detecting a signal associated with the monomethine cyanine dye or salt thereof. In some embodiments, the double-intercalating monomethine cyanine dye is dimeric. In some embodiments, the double-intercalating monomethine cyanine dye is a homodimer. In some embodiments, the contacting comprises hybridization of the nucleic acid probe to a target sequence comprised in or associated with the RCP at the location in the biological sample. In some embodiments, the RCP is generated from a circular template at the location in the biological sample. In some embodiments, the circular template is a circular probe that directly or indirectly binds to the target nucleic acid at the location in the biological sample. In some embodiments, the circular template is generated at the location in the biological sample. In some embodiments, the target nucleic acid is an RNA, and the circular template is generated by transcribing the RNA in the biological sample generate a molecule comprising a cDNA of the RNA and circularizing the molecule comprising the cDNA. In some embodiments, the circular template is generated by contacting the biological sample with a circularizable probe or probe set that directly or indirectly binds to the target nucleic acid at the location in the biological sample, and circularizing the circularizable probe or probe set.

In some embodiments, provided herein are systems comprising: a) a cell or tissue sample comprising a target nucleic acid; b) a circular template, wherein the circular template comprises a hybridization region complementary to the target nucleic acid, wherein the circular template comprises a priming sequence complementary to a primer; and c) a nucleic acid probe non-covalently associated with a monomethine cyanine dye or a salt thereof, wherein the nucleic acid probe comprises a sequence complementary to a product of the circular template. In some embodiments, the system further comprises a circularizable probe or probe set. In some embodiments, the circularizable probe or probe set is used to form the circular template. In some embodiments, the system further comprises a ligase for forming the circular template. In some embodiments, the system further comprises a reaction mixture comprising a polymerase and a mixture of free nucleotides including the four canonical bases: adenine, thymine, guanine, and cytosine. In some embodiments, the system further comprises the primer. In some embodiments, the cell or tissue sample comprises the primer. In some embodiments, the reaction mixture comprises the primer.

In some embodiments, the target nucleic acid is an endogenous nucleic acid. In some embodiments, the target nucleic acid is a cellular nucleic acid. In some embodiments, the cellular nucleic acid is genomic DNA. In some embodiments, the cellular nucleic acid is RNA. In some embodiments, the target nucleic acid is a reporter oligonucleotide comprised on a labeling agent, wherein the labeling agent is bound to an analyte (e.g., a target analyte) in the biological sample. In some embodiments, the signal associated with the monomethine cyanine dye is a fluorescence signal. In some embodiments, the detecting comprises using fluorescence microscopy. In some embodiments, the biological sample is a fresh tissue sample. In some embodiments, the biological sample is a frozen tissue sample. In some embodiments, the biological sample is a fixed tissue sample. In some embodiments, the biological sample is a fresh-frozen tissue sample. In some embodiments, the biological sample is a formalin-fixed paraffin-embedded tissue section. In some embodiments, the biological sample is derived from a human. In some embodiments, the biological sample is derived from a human with a disease or condition. In some embodiments, the biological sample is derived from a non-human mammal.

### A. Monomethine Cyanine Dyes

Disclosed herein in some aspects are monomethine cyanine dyes or salts thereof. In some aspects, a monomethine cyanine dye or salt thereof associates with a nucleic acid probe. In some embodiments, the monomethine cyanine dye or salt thereof non-covalently associated with the nucleic acid probe. In some embodiments, the non-covalent association is electrostatic binding between the monomethine cyanine dye or salt thereof and one or more bases of the nucleic acid probe. In some embodiments, the electrostatic binding is the result of 7-stacking with the one or more bases of the nucleic acid probe. In some embodiments, the one or more bases of the nucleic acid probe are poly(dA). In some embodiments, the non-covalent association is intercalation. In some embodiments, the intercalation is with a double-stranded form comprised on the nucleic acid probe. In some embodiments, the intercalation is with the minor groove of the double-stranded form comprised on the nucleic acid probe. In some embodiments, the double-stranded form is a stem-loop/hairpin form.

In some aspects, the monomethine cyanine dye or salt thereof comprise a cyanine chromophore. In some embodiments, the cyanine chromophore comprises a monomethine chain. In some embodiments, the monomethine cyanine dye or salt thereof comprises —C═C— bonds that connect N-containing end heterocycles. In some embodiments, the monomethine cyanine dye or salt thereof is symmetrical. In some embodiments, the monomethine cyanine dye or salt thereof is asymmetrical.

In some embodiments, the monomethine cyanine dye comprises a general structure of

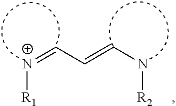

wherein N is an N-containing heterocycle, R₁ is a first substituent, and R₂ is a second substituent. In some embodiments, the first substituent and/or the second substituent is any moiety that contributes to the final structure and fluorescence capacity of the monomethine cyanine dye, such as an aromatic ring. For example, in some embodiments, the N-containing heterocycle further comprises a variable position X. In some embodiments, the variable position comprises an atom or atoms selected from the group consisting of C(CH₃)₂, NR₁, O, S, Se, and Te. In some embodiments, the variable position X is O. In some embodiments, the variable position X is S.

In some embodiments, the monomethine cyanine dye or salt thereof is derived from a precursor compound using a reaction scheme. In some embodiments, the precursor compound is selected from the group consisting of thioalkyl derivatives, 2-iminobenzo-thiazoline, sulfobetaine salt of N-alkyl heterocycle, 2- or 4-chloroquinoline derivatives, 3-phenyl-2H-1,4-benzothiazine and indole-3-carboxaldehyde, methyl-pyridinium iodide, and thiazole orange derivative. Example reaction schemes for generating monomethine cyanine dyes from the disclosed precursor compounds are described, for instance, in Table 1 of Pronkin and Tatikolov, “Fluorescent Probes for Biomacromolecules Based on Monomethine Cyanine Dyes”, Chemosensors, (2023), 11:280, 1-35, the content of which is herein incorporated by reference in its entirety.

In some embodiments, the monomethine cyanine dye is a monomeric monomethine cyanine dye. In some embodiments, the monomeric monomethine cyanine dye associated with a nucleic probe provided in single-stranded form. Without being bound by theory, the monomeric monomethine cyanine dye can bind electrostatically to one or more bases comprised on the single-stranded nucleic acid probe. In some embodiments, the monomeric monomethine cyanine dye is derived from thiazole orange. In some embodiments, the monomeric monomethine cyanine dye is selected from the group consisting of YO-Pro-1, TO-Pro-1, PO-Pro-1, BO-Pro-1, and TO-Pro-3.

In some embodiments, the monomeric monomethine cyanine dye has the structure:

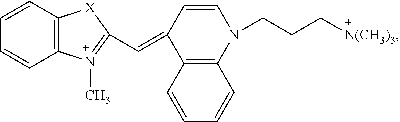

wherein X is O or S. In some embodiments, the X is O and in such embodiments, the monomeric monomethine cyanine dye is YO-Pro-1. In some embodiments, the X is S, and in such embodiments the monomeric monomethine cyanine dye is TO-Pro-1.

In some embodiments, the monomeric monomethine cyanine dye has the structure:

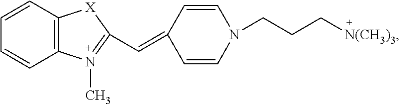

wherein X is O or S. In some embodiments, the X is O, and in such embodiments the monomeric monomethine cyanine dye is PO-Pro-1. In some embodiments, the X is S, and in such embodiments the monomeric monomethine cyanine dye is BO-Pro-1.

In some embodiments, the monomeric monomethine cyanine dye as the structure:

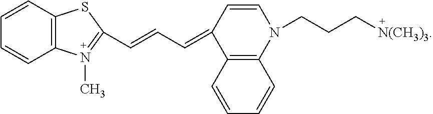

In some embodiments, the monomeric monomethine cyanine dye is TO-Pro-3.

In some embodiments, the monomethine cyanine dye is a dimeric monomethine cyanine dye. In some embodiments, the dimeric monomethine cyanine dye associates non-covalently with a nucleic probe provided in partially double-stranded form. In some embodiments, the dimeric monomethine cyanine dye associates non-covalently with a hairpin/stem-loop form of the nucleic acid probe. Without being bound by theory, the dimeric monomethine cyanine dye can intercalate the double-stranded structure (e.g., the stem-loop and/or hairpin) of the nucleic acid probe.

In some embodiments, the dimeric monomethine cyanine dye is derived from thiazole orange. In some embodiments, the dimeric monomethine cyanine dye is a homodimer comprising two identical monomeric monomethine cyanine dye moieties. In some embodiments, the dimeric monomethine cyanine dye is a heterodimer comprising two different monomeric cyanine dye moieties. In some embodiments, the dimeric monomethine cyanine dye is selected from the group consisting of YOYO-1, TOTO-1, POPO-1, BOBO-1, and TOTO-3.

In some embodiments, the dimeric monomethine cyanine dye has a structure of formula:

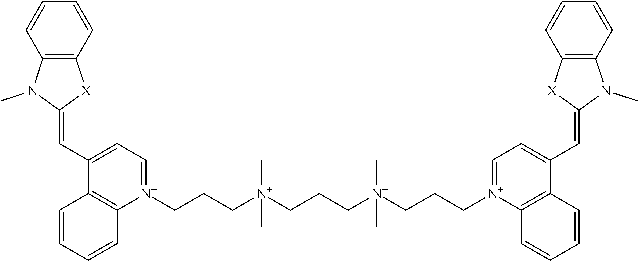

wherein X is S or O. In some embodiments, the X is O, and in such embodiments the dimeric monomethine cyanine dye is YOYO-1. In some embodiments, the X is S, and in such embodiments, the monomeric monomethine cyanine dye is TOTO-1.

In some embodiments, the dimeric monomethine cyanine dye has a structure of formula:

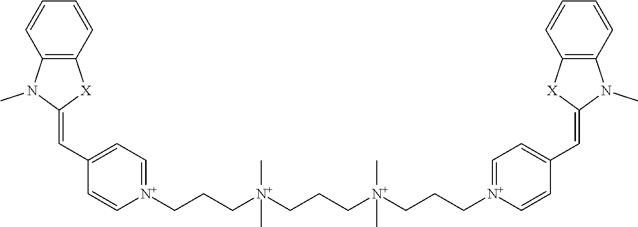

wherein X is S or O. In some embodiments, the X is O. In such embodiments, the dimeric monomethine cyanine dye is POPO-1. In some embodiments, the X is S, and in such embodiments, the monomeric monomethine cyanine dye is BOBO-1.

In some embodiments, the dimeric monomethine cyanine dye has the structure:

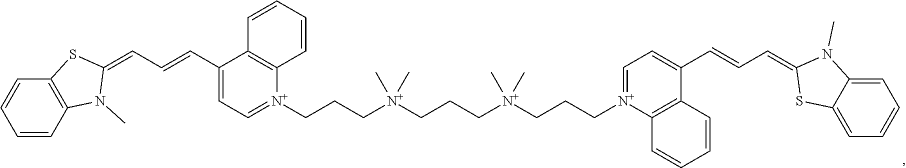 [SMILES: CN1/C(=C/C=C/c2cc[n+](CCC[N+](C)(C)CCC[N+](C)(C)CCC[n+]3ccc(/C=C/C=C4/Sc5ccccc5N4C)c4ccccc43)c3ccccc23)Sc2ccccc21]

which is the dimeric monomethine cyanine dye TOTO-3.

### B. Nucleic Acid Probes

Disclosed herein in some aspects are nucleic acid probes that hybridize directly or indirectly to a target nucleic acid in a biological sample. In some embodiments, the nucleic acid probes are associated with a monomethine cyanine dye or salt thereof or derivative thereof. In some embodiments, the nucleic acid probes are contacted with the biological sample comprising the target nucleic acid. In some embodiments, the biological sample is a cell, a tissue sample, or a tissue section, or any one biological sample disclosed in Section III.A. In some aspects, the nucleic acid probe comprises a sequence complementary to a target sequence comprised on the target nucleic acid or a product thereof. In some embodiments, the nucleic acid probe comprises a sequence complementary to a probe associated with the target nucleic acid (e.g., a circular probe or circularizable probe or probe set). In some embodiments, the nucleic acid probe comprises a sequence complementary to a rolling circle amplification product (RCP) associated with the target nucleic acid. In some embodiments, the RCP is derived from a circular template directly or indirectly associated with the target nucleic acid. In some embodiments, the nucleic acid probe hybridizes directly or indirectly to the target nucleic acid at a complementary sequence. In some embodiments, association and/or hybridization of the nucleic acid probe to the target nucleic acid results in association of a signal to the target nucleic acid for subsequent detection of the target nucleic acid within the biological sample. In some embodiments, the signal is a fluorescent signal derived from the monomethine cyanine dye associated with the nucleic acid probe.

The nucleic acid probes may comprise any of a variety of entities that can hybridize to a nucleic acid, typically by Watson-Crick base pairing, such as DNA, RNA, LNA, PNA, etc. In some embodiments, the nucleic acid probe comprises DNA. In some embodiments, the nucleic acid probe is DNA. In some embodiments, the nucleic acid probe comprises RNA. In some embodiments, the nucleic acid probe is RNA. In some aspects, the nucleic acid probe comprises a sequence (e.g., hybridization region such as a target recognition sequence) that directly or indirectly binds to at least a portion of the target nucleic acid (e.g., a target RNA). In some instances, the nucleic acid probe binds to a specific target nucleic acid (e.g., an mRNA, or other nucleic acids as discussed herein). In some embodiments, the nucleic acid probe binds to a probe or product thereof associated with a target nucleic acid (e.g., a circular probe or circularizable probe or product thereof). In some embodiments, the nucleic acid probe binds to a reporter oligonucleotide comprised on a labeling agent, wherein the reporter oligonucleotide is the target nucleic acid. In some embodiments, the nucleic acid probe binds to a probe or probe set, or product thereof, associated with a reporter oligonucleotide of a labeling agent, wherein the reporter oligonucleotide is the target nucleic acid.

In some embodiments, more than one type of nucleic acid probe is contacted with the sample. In some instances, the biological sample is contacted with a first nucleic acid probe and a second nucleic acid probe, wherein the first nucleic acid probe is associated with a first monomethine cyanine dye or salt thereof, and the second nucleic acid probe is associated with a second monomethine cyanine dye or salt thereof. In some embodiments, the biological sample is contacted with the first nucleic acid probe and the second nucleic acid probe simultaneously. In some embodiments, the biological sample is contacted with the first nucleic acid probe and the second nucleic acid probe in sequential order. In some embodiments, the first nucleic acid probe comprises a sequence complementary to a first target sequence, and the second nucleic acid probe comprises a sequence complementary to a second target sequence. In some embodiments, the first target sequence and the second target sequence are different nucleic acid sequences comprised on a common target molecule, such as a single target nucleic acid or a single probe or product thereof. In some embodiments, the first target sequence is comprised on a first target molecule (e.g., a first target nucleic acid or probe or product thereof associated with the first target nucleic acid) and the second target sequence is comprised on a second target molecule (e.g., a second target nucleic acid or probe or product thereof associated with the second target nucleic acid). In some embodiments, the method further comprises contacting the biological sample with a third nucleic acid probe and a fourth nucleic acid probe, wherein the third nucleic acid probe is associated with a third monomethine cyanine dye or salt thereof, and the fourth nucleic acid probe is associated with a fourth monomethine cyanine dye or salt thereof. In some embodiments, the method allows for multiplexed detection of a single target nucleic acid using a plurality of nucleic acid probes, wherein each nucleic acid probe of the plurality of nucleic acid probes is labeled with a different monomethine cyanine dye. In some embodiments, the method allows for multiplexed detection of a plurality of target nucleic acids using a plurality of nucleic acid probes, wherein each nucleic acid probe of the plurality of nucleic acid probes associates with a different target nucleic acid of the plurality of target nucleic acids, and each nucleic acid probe of the plurality of nucleic acid probes is labeled with a different monomethine cyanine dye.

### (i) Providing Nucleic Acid Probes

In some embodiments, provided herein are methods and compositions for generating nucleic acid probes comprising nucleic acid molecules and monomethine cyanine dyes or derivatives thereof or salts thereof. In some embodiments, the nucleic acid probe is generated by contacting a nucleic acid molecule with a monomethine cyanine dye or a derivative thereof or a salt thereof. In some embodiments, the contacting results in association between the nucleic acid molecule and the monomethine cyanine dye, thereby forming the nucleic acid probe. In some embodiments, the association is a non-covalent interaction. In some embodiments, the non-covalent interaction is intercalation of the monomethine cyanine dye with the nucleic acid molecule, wherein the nucleic acid molecule is double-stranded DNA molecule. In some embodiments, the non-covalent interaction is binding of the monomethine cyanine dye to a nucleotide comprised in the nucleic acid molecule. In some embodiments, the nucleotide is adenine (dA) or guanine (dG). In some embodiments the non-covalent interaction is binding of the monomethine cyanine dye to poly(dA) and/or poly(dG) comprised in the nucleic acid molecule. In some embodiments, the monomethine cyanine dye is monomeric. In some embodiments, the monomethine cyanine dye is dimeric. In some embodiments, the monomethine cyanine dye does not dissociate from the nucleic acid probe during use in an assay, for example when contacted with a biological sample and/or hybridized to a target sequence.

In some embodiments, the nucleic acid probe comprises a nucleic acid molecule capable of hybridizing to a complementary target sequence. In some embodiments, the nucleic acid probe comprises substantially complementary or complementary nucleic acid sequences to a target nucleic acid sequence. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In some embodiments, the length of the nucleic acid probe enables stable Watson-Crick base-pairing with a sufficiently or fully complementary target nucleic acid sequence. In some embodiments, the nucleic acid probe comprises a nucleotide length capable of forming secondary structures, for instance stem-loop or hairpin structures. Example secondary structures of nucleic acid probes are outlined in Section II.B.(iii). In some embodiments, the nucleic acid probe is at least 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides in length, or at least about 120 nucleotides in length. In some embodiments, the nucleic acid probe is 10 to 100 nucleotides in length, 10 to 50 nucleotides in length, 20 to 100 nucleotides in length, 20 to 200 nucleotides in length, or 50 to 200 nucleotides in length. In some embodiments, the nucleic acid probe is about 20 nucleotides in length. In some embodiments, the nucleic acid probe is about 30 nucleotides in length. In some embodiments, the nucleic acid probe is about 40 nucleotides in length. In some embodiments, the nucleic acid probe is about 50 nucleotides in length. In some embodiments, the nucleic acid probe is about 100 nucleotides in length. In some embodiments, the nucleic acid probe is provided in a fully single-stranded form and is about 20 nucleotides in length, about 30 nucleotides in length, about 40 nucleotides in length, about 50 nucleotides in length, or about 100 nucleotides in length. In some embodiments, the nucleic acid probe is provided in stem-loop form and is about 60 nucleotides in length, about 70 nucleotides in length, about 80 nucleotides in length, about 90 nucleotides in length, about 100 nucleotides in length, about 110 nucleotides in length, or about 120 nucleotides in length.

In some embodiments, forming the nucleic acid probe comprises contacting the nucleic acid molecule with the monomethine cyanine dye comprises incubation in vitro. In some embodiments, contacting the nucleic acid probe with the monomethine cyanine dye comprises incubating the nucleic acid probe in a solution comprising the monomethine cyanine dye or the salt thereof. In some embodiments, the incubation/contacting is for about 30 seconds to about 1 hour. In some embodiments, the incubation/contacting is for about 30 seconds, about 1 minute, about 2 minutes, about 3 minutes, about 4 minutes, about 5 minutes, about 7 minutes, about 10 minutes, about 15 minutes, about 20 minutes, about 30 minutes, about 40 minutes, about 45 minutes, or about 60 minutes. In some embodiments, the incubation/contacting is for about 30 seconds to about 2 minutes. In some embodiments, the incubation/contacting is for about 30 seconds. In some embodiments, the incubation/contacting is for about 2 minutes. In some embodiments, the incubation/contacting is for about 5 minutes. In some embodiments, the incubation/contacting is for about 10 minutes. In some embodiments, the incubation/contacting is for about 15 minutes. In some embodiments, the incubation/contacting is for about 20 minutes. In some embodiments, the incubation/contacting is for about 30 minutes. In some embodiments, the incubation/contacting is for about 45 minutes. In some embodiments, the incubation/contacting is for about 60 minutes/1 hour.

In some embodiments, the nucleic acid molecule is provided in a buffer solution. The buffer solution can be any standard, aqueous solution resistant to pH changes, for example phosphate buffered saline (PBS) or Hank's Balanced Salt Solution (HBSS). In some embodiments, the buffer solution is a hybridization buffer. In some embodiments, the monomethine cyanine dye or salt thereof is provided in a stock solution. In some embodiments, the stock solution is an organic solvent such as dimethyl sulfoxide (DMSO). In some embodiments, a volume of the stock solution of the monomethine cyanine dye or salt thereof is mixed with the buffer solution comprising the nucleic acid molecule. In some embodiments, the mixing is facilitated by gentle pipetting, tapping, and/or vortexing.

In some embodiments, the stock of the monomethine cyanine dye or salt thereof is provided at a concentration of about 10 μM, about 100 μM, about 200 μM, about 300 μM, about 400 μM, about 500 μM, about 750 μM, about 1 mM, about 1.5 mM, about 2 mM, about 3 mM, about 5 mM, or about 10 mM. In some embodiments, the stock of the monomethine cyanine dye or salt thereof is provided in a concentration of about 1 mM. In some embodiments, the monomethine cyanine dye or salt thereof is added to the nucleic acid molecule at a final concentration of about 0.1 μM, about 0.2 μM, about 0.3 μM, about 0.4 μM, about 0.5 μM, about 0.6μM, about 0.7 μM, about 0.8 μM, about 0.99 μM, about 1.0 μM, about 1.1 μM, about 1.2 μM, about 1.3 μM, about 1.4 μM, about 1.5 μM, about 1.6 μM, about 1.7 μM, about 1.88 μM, about 1.9 μM, about 2 μM, about 3 μM, about 4 μM, about 5 μM, about 6 μM, about 7 μM, about 8 μM, about 9 μM, or about 10 μM. In some embodiments, the monomethine cyanine dye or salt thereof is added to the nucleic acid molecule at a final concentration of about 1. OM.

In some embodiments, the nucleic acid molecule used to form the nucleic acid probe is provided in a concentration sufficient for efficient association with the monomethine cyanine dye or salt thereof. In some embodiments, the nucleic acid molecule used to form the nucleic acid probe is provided at a concentration of about 1 μM, about 5 μM, about 10 μM, about 25 μM, about 50 μM, about 75 μM, about 100 μM, about 200 μM, about 300 μM, about 400 μM, about 500 μM, about 750 μM, or about 1 mM. In some embodiments, the nucleic acid molecule is provided at a concentration of about 100 μM.

In some embodiments, the mixture is incubated to facilitate association between the nucleic acid molecule and monomethine cyanine dye or salt thereof to form the nucleic acid probe. In some embodiments, the mixture is protected from light, for example by wrapping a tube containing the mixture in tinfoil. In some embodiments, the mixture is incubated for at least 10 seconds, at least 20 seconds, at least 30 seconds, at least 1 minute, at least 3 minutes, at least 5 minutes, at least 10 minutes, at least 20 minutes, at least 30 minutes, at least 45 minutes, or at least 60 minutes. In some embodiments, the mixture is incubated for at least 1 hour, at least 2 hours, at least 3 hours, at least 4 hours, or at least 5 hours. In some embodiments, the mixture is incubated at room temperature. In some embodiments, the mixture is incubated at 37° C. In some embodiments, the mixture is incubated at 4° C. In some embodiments, the mixture is stored at 4° C. for an extended period of time following formation of the nucleic acid probe. Formation of the nucleic acid probe is schematized, for example, in FIG. 1A, which shows incubation of an unlabeled nucleic acid molecule (101) with an example monomethine cyanine dye (MCD) Po-Pro-1 (102) to form a nucleic acid probe (103) non-covalently associated with the MCD.

### (ii) Single-Stranded Nucleic Acid Probe

In some embodiments, the nucleic acid probe is provided in single-stranded form. In some embodiments, the nucleic acid probe comprises a single-stranded nucleic acid molecule. In some embodiments, the nucleic acid probe is ssDNA. In some embodiments, the nucleic acid probe is ssRNA. In some embodiments, the nucleic acid probe does not comprise self-complementarity. In some embodiments, the nucleic acid probe does not form any stable secondary structures.

In some embodiments, the nucleic acid probe is a single-stranded nucleic acid probe associated with a monomethine cyanine dye or derivative thereof or salt thereof. The monomethine cyanine dye associated with the single-stranded nucleic acid probe can be any one of the example dyes described in Section II.A. In some embodiments, the monomethine cyanine dye is monomeric. Non-covalent association of a monomeric monomethine cyanine dye with a nucleic acid molecule may result from a number of interaction such as direct binding of dG and dA bases, in particular stretches of poly(dA) or poly(dG), within the molecule or intercalation with certain stretches of bases, such as poly(dA). Without being bound by theory, monomeric monomethine cyanine dyes may intercalate with poly(dA) on single-stranded nucleic acid molecules through π-stacking with the quinoline ring between two dA bases. Further details into the mechanism of association between monomeric monomethine cyanine dyes and single-stranded nucleic acid molecules (e.g., ssDNA nucleic acid probes) is found in Mikelsons et al., Photochem. Photobiol. Sci., (2005), 4, 798-802, the content of which is herein incorporated by references in its entirety.

In some embodiments, the method comprises binding a single-stranded nucleic acid probe to a target nucleic acid at a target sequence complementary to the nucleic acid probe. In some embodiments, the single-stranded nucleic acid probe comprises sufficient or full complementarity to the target sequence. In these instances, a substantial portion or the entire length of the single-stranded nucleic acid probe may hybridize to the target sequence.

In other embodiments, only one or more portions of the single-stranded nucleic acid probe is complementary to the target sequence, while the other portions are not complementary to the target sequence. In some embodiments, the one or more portions comprising complementarity hybridize to the target sequence, while the other portions do not hybridize to the target sequence. For example, the single-stranded nucleic acid probe may comprise a 5′-complementary arm and a 3′ complementary arm at each end, wherein both arms are about 5 to about 20 nucleotides in length, and a non-complementary middle portion (e.g., from about 20 to about 50 nucleotides in length). In some embodiments, the structure of the single-stranded nucleic acid probe comprises from 5′ to 3′: 5′—complementary region 1—non-complementary region—complementary region 2-3′, wherein the non-complementary region does not form a secondary structure. In this example, the 5′-complementary arm may hybridize to a first portion of the target sequence, and the 3′ complementary arm may hybridize to a second portion of the target sequence, wherein the first portion and the second portion of the target sequence are next to or adjacent to each other.

(iii) Partially Double-Stranded Nucleic Acid Probe

In some embodiments, the nucleic acid probe is provided in partially double-stranded form. In some embodiments, the nucleic acid probe comprises and/or is DNA. In some embodiments, the nucleic acid probe comprises and/or is RNA. In some embodiments, the nucleic acid probe comprises a first portion comprising a double-stranded secondary structure, and a second portion comprising a target recognition sequence complementary to a target sequence comprised on the target nucleic acid. In some embodiments, the double-stranded secondary structure is a hairpin and/or stem-loop structure. In some embodiments, the nucleic acid probe associates with the monomethine cyanine dye primarily at the double-stranded secondary structure. In some embodiments, the monomethine cyanine dye is dimeric. In some embodiments, the monomethine cyanine dye is a homodimer. Without being bound by theory, association of dimeric monomethine cyanine dye with the double-stranded secondary structure is facilitated by intercalation of the dye within the double-stranded portion, for example within the minor groove of dsDNA. The monomethine cyanine dye associated with the single-stranded nucleic acid probe can be any one of the example dyes described in Section II.A.

In some embodiments, the target recognition sequence of a nucleic acid probe is positioned 3′ to the double-stranded secondary structure in the nucleic acid probe. In some embodiments, the target recognition sequence of a nucleic acid probe is positioned 5′ to the double-stranded secondary structure in the nucleic acid probe. In some embodiments, the target recognition sequence comprises a sequence that is substantially complementary to a portion of a target nucleic acid (e.g., a target sequence). In some embodiments, the target recognition sequence and the target sequence are at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

In some embodiments, the partially double-stranded nucleic acid probe comprises a hairpin and/or stem-loop structure. For example, the nucleic acid probe comprises; the target recognition sequence, a stem sequence, a loop sequence, and a complement of the stem sequence to form the stem loop structure. Without being bound by theory, complementarity between the stem sequence and the complement of the stem sequence can facilitate Watson-Crick base-pairing that enables formation of a hairpin/stem-loop structure. In some cases, the nucleic acid probe comprises one or more additional sequences outside of the stem and loop sequences, such as the target recognition sequence. In some embodiments, the extension probe comprises from 5′ to 3′: 5′—the complement of the stem sequence—the loop domain—the stem sequence—the target recognition sequence—3′. In some embodiments, the probe comprises from 5′ to 3′: 5′—the target recognition sequence—the complement of the stem sequence—the loop domain—the stem sequence—3′. In some embodiments, the stem sequence and the complement of the stem sequence each are about 20 to 50 in length or about 35 to 45 nucleotides in length. In some embodiments, the loop sequence is 4 to 8 nucleotides in length. In some embodiments, the target recognition sequence is about 10 to 20 nucleotides in length.

For example, FIG. 4A shows labeling of a hairpin-form nucleic acid probe (401) using an example dimeric monomethine cyanine dye (MCD) (402) to form a labeled hairpin-form nucleic acid probe used for detection (403). FIG. 4B shows detection of an RCP (404) using the labeled hairpin-form nucleic acid probe (403).

Providing nucleic acid probes in partially double-stranded form may significantly increase brightness of the signal associated with the probe. Without being bound by theory, decoupling of the dye-binding domain of the nucleic acid probe with the domain that binds the target nucleic acid may provide more space dye intercalation, thereby increasing the fluorescent signal associated with a single nucleic acid probe, thereby helping increase the sensitivity of in situ detection assays.

C. Use of a Nucleic Acid Probe Associated with a Monomethine Cyanine Dye

### (i) In Situ Assays Comprising Nucleic Acid Probes

In some aspects, the provided methods involve analyzing one or more sequences present in the target nucleic acid present in a biological sample using any one of the nucleic acid probes described heretofore. In some aspects, the provided methods involve analyzing one or more sequences present in the probes or probe sets or products thereof (e.g., rolling circle amplification products thereof) comprised in a biological sample. In some embodiments, the analyzing comprises contacting the biological sample with a nucleic acid probe that directly or indirectly associated with the target nucleic acid. In some embodiments, the analyzing comprises detecting a signal associated with the nucleic acid probe. For example, FIG. 1B shows detection (105) of a rolling circle amplification product (RCP; 104) using the nucleic acid probe (103) non-covalently associated with a monomethine cyanine dye or salt thereof.

In some embodiments, the detecting is performed at one or more locations in the biological sample. In some embodiments, the locations are the locations of RNA transcripts in the biological sample. In some embodiments, the locations are the locations at which the probes or probe sets hybridize to the RNA transcripts in the biological sample, and are optionally ligated and amplified by rolling circle amplification. In some embodiments, the method comprises contacting a biological sample with a nucleic acid probe, wherein the nucleic acid probe is non-covalently associated with a monomethine cyanine dye or a salt thereof, wherein the contacting results in the nucleic acid probe binding directly or indirectly to a target nucleic acid at a location in the biological sample.

In some embodiments, the target nucleic acid is an endogenous nucleic acid (e.g., a DNA or an RNA) comprised at the location in the biological sample. In some embodiments, a product is derived from the target nucleic acid at a location in the biological sample, and the nucleic acid probe binds to the product of the target nucleic acid, thereby indirectly binding to the target nucleic acid at the location in the biological sample. In some embodiments, the target nucleic acid is a reporter oligonucleotide comprised on a labeling agent associated with an analyte. In some embodiments, a probe or probe set (e.g., a circularizable probe or probe set) associates directly or indirectly with the target nucleic acid at a location in the biological sample, and the nucleic acid probe binds to the probe or probe set, thereby indirectly binding to the target nucleic acid at the location in the biological sample. In some embodiments, a probe or probe set (e.g., a circularizable probe or probe set) associates directly or indirectly with the target nucleic acid at a location in the biological sample and a product is generated from the probe or probe set associated with the target nucleic acid, and the nucleic acid probe binds to the product of the probe or probe set, thereby indirectly binding to the target nucleic acid at the location in the biological sample.

In some embodiments, the analyzing comprises a plurality of repeated cycles of hybridization, detection, and removal of probes (e.g., nucleic acid probes, or intermediate probes that bind to nucleic acid probes) that bind to the target nucleic acid, to the primary probe or probe set hybridized to the target nucleic acid, or to a rolling circle amplification product generated from the probe or probe set hybridized to the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, all of which are incorporated herein by reference in their entireties. Nucleic acid probes can be useful for detecting multiple target nucleic acids and be detected in one or more hybridization cycles (e.g., sequential hybridization assays, or sequencing by hybridization).

In some embodiments, the analyzing comprises binding an intermediate probe directly or indirectly to the primary probe or probe set, binding a nucleic acid probe directly or indirectly to a detection region of the intermediate probe, and detecting a signal associated with the nucleic acid probe. In some embodiments, the intermediate probe is associated with the target nucleic acid, and binding of the nucleic acid probe to the intermediate probe results in indirect association/binding of the nucleic acid probe to the target nucleic acid. In some embodiments, the analyzing comprises detecting a rolling circle amplification product (RCP) generated using a circular or circularized primary probe or probe set as a template. In some embodiments, the analyzing comprises detecting a rolling circle amplification product (RCP) generated using a circular or circularized probe or probe that binds to a primary probe or probe set as a template. In some embodiments, the analyzing comprises binding an intermediate probe directly or indirectly to the RCP, binding a nucleic acid probe directly or indirectly to a detection region of the intermediate probe, and detecting a signal associated with the nucleic acid probe. In some embodiments, the method comprises performing one or more wash steps to remove unbound and/or nonspecifically bound intermediate probe molecules from the primary probes or the products of the primary probes.

In some embodiments, the analyzing comprises: detecting signals associated with nucleic acid probes that are hybridized to barcode regions or complements thereof in the primary probe or probe set or a product thereof (e.g., an RCP); and/or detecting signals associated with nucleic acid probes that are hybridized to intermediate probes which are in turn hybridized to the barcode regions or complements thereof. In some embodiments, the signals associated with the nucleic acid probes are generated by or derived from one or more associated monomethine cyanine dyes or derivatives thereof or salts thereof.

An example, non-limiting workflow for in situ analysis of biological samples is shown in FIG. 5. First, in 501 a biological sample (e.g. a tissue section) is contacted with a circularizable probe that hybridizes to a target nucleic acid of interest at a location in the biological sample, for example an endogenous mRNA. The biological sample is washed with one or more washing steps (e.g., using a buffer such as a hybridization buffer or PBS) to remove any circularizable probes that are not hybridized to the target nucleic acid (502). The circularizable probes hybridized to the target nucleic acid are then circularized using the target nucleic acid as a template (e.g., through ligation) to form a circular template (503). Use of the target nucleic acid as a template for a reaction such as ligation may add a layer of specificity to the assay, as unbound probes should not be circularized. Next, as described in 504, rolling circle amplification is performs on the circular template to form a rolling circle amplification product (RCP) comprised at the location in the biological sample. Rolling circle amplification can be performed as described, for example, in Section II.C.(ii) below. For example, rolling circle amplification may comprise an additional step of contacting the biological sample with a primer and a polymerase with strand-displacement capacity, and incubating the biological sample in conditions amendable to polymerization using the circular template as a template. After the RCP is generated, the biological sample is contacted with a nucleic acid probe non-covalently associated with a monomethine cyanine dye (505). The nucleic acid probe hybridizes to at least a portion of a complementary target sequence comprised on the RCP. Next, the biological sample is optionally washed to remove any unbound nucleic acid probes from the sample (506). This may help reduce background signal generated from the unbound nucleic acid probe. Finally, the biological sample is imaged to detect a signal (e.g., a fluorescence signal) associated with the monomethine cyanine dye, thereby detecting the target nucleic acid at the location in the biological sample (507).

### (ii) Generation of Probe Products

In some embodiments, provided herein are methods and compositions for analyzing a target nucleic acid in a biological sample, wherein the target nucleic acid is or is associated with one or more products of an endogenous analyte and/or a labeling agent in a biological sample. In some embodiments, the methods comprise association of a nucleic acid probe with the target nucleic acid, wherein the analyzing comprises detection of a signal associated with a monomethine cyanine dye comprised on the nucleic acid probe. In some embodiments, the target nucleic acid is an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof. In some embodiments, a labeling agent that directly or indirectly binds to an analyte in the biological sample is analyzed, wherein the labeling agent comprises the target nucleic acid. In some embodiments, the target nucleic acid is a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample.

### (a) Hybridization

In some embodiments, a hybridization product comprising the pairing of substantially complementary or complementary nucleic acid sequences within two different molecules is analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of nucleic acid hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Exemplary probes or probe sets, including barcoded probes or probe sets, may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. In some embodiments, the probe or probe set is a circularizable probe or probe set. In some embodiments, the probe is a circular probe. In some embodiments, the probe or probe set is used to generate a circular template through any of the means described below in Section II.C.(ii).(b). In some embodiments, the circular probe is a circular template. The specific probe or probe set design can vary.

### (b) Ligation

In some embodiments, a ligation product of an endogenous analyte and/or a labeling agent can be analyzed. In some embodiments, the ligation product is formed between two or more endogenous analytes. In some embodiments, the ligation product is formed between two or more labeling agents. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe is indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the method comprises generation of a circular template from a probe or probe set. In some embodiments, the probe or probe set is a circularizable probe or probe set. In some embodiments, the circularizable probe or probe set is ligated to form the circular template. In some embodiments, the method comprises contacting the biological sample with a circular probe, wherein the circular probe is a circular template. In some embodiments, the circular template is used as a template for amplification, for instance using the methods described in Section II.C.(ii).(c).

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some embodiments, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) is analyzed. In some embodiments, the primer extension product is a product derived from a template. In some embodiments, the template is a circular template, and the primer extension product is a rolling circle amplification product (RCP).

A primer is generally a single-stranded nucleic acid sequence having a 3′ end that, in some embodiments, is used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. In some embodiments, enzymatic extension is performed by an enzyme including, but not limited to, a polymerase and/or a reverse transcriptase.

In some embodiments, a product of an endogenous analyte and/or a labeling agent is an amplification product of one or more polynucleotides, for instance, a circular probe or circularizable probe or probe set. In some embodiments, the product is an amplification product of a circular template. In some embodiments, the circular template is derived from a circular probe or a circularizable probe or probe set. In some embodiments, the circular template is derived from circularization of an endogenous nucleic acid. In some embodiments, the amplifying is achieved by performing rolling circle amplification (RCA). In some embodiments, an endogenous nucleic acid or fragment thereof hybridized to the circular probe or circularized probe is used to prime amplification. In some embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, amplification of a circular template, a circular probe or circularizable probe or probe set is primed by the target RNA. The target RNA can optionally be immobilized in the biological sample. In some embodiments, the target RNA is cleaved by an enzyme (e.g., RNase H). In some embodiments, the target RNA is cleaved at a position downstream of the target sequences bound to the circular probe or circularizable probe or probe set. In some aspects, the methods disclosed herein allow targeting of RNase H activity to a particular region in a target RNA that is adjacent to or overlapping with a target sequence for a probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe or probe set by about 1 to about 20 nucleotides or by about 8 to about 10 nucleotides. The cleaved target RNA itself can then be used to prime RCA of the circular probe generated from a circularizable probe or probe set (e.g., target-primed RCA). In some cases, a plurality of nucleic acid oligonucleotides can be used to perform target-primed RCA for a plurality of different target RNAs.

In any of the embodiments herein, the biological sample is contacted with the RNase H (and optionally with the nucleic acid oligonucleotide) before or during formation of the circularized gap-filled first probe or probe set. In some embodiments, the biological sample is contacted with the oligonucleotide and with the RNase H simultaneously or sequentially (in either order) before contacting the sample with the probe or probe set. In any of the embodiments herein, the biological sample can be contacted with the RNase H (and optionally with the nucleic acid oligonucleotide) after formation of the circularized probe or probe set. In any of the embodiments herein, the RNase H comprises an RNase H1 and/or an RNAse H2. In some embodiments, RNase inactivating agents or inhibitors are added to the sample after cleaving the target RNA.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., U.S. Pat. Nos. 6,054,274, 6,291,187, 6,323,009, 6,344,329 and 6,368,801, each of which are herein incorporated by reference in their entireties). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides are added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) are anchored to a polymer matrix. For example, the polymer matrix can be a hydrogel. In some embodiments, one or more of the polynucleotide probe(s) is modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using an RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template and comprises complementary copies of the RCA template. The RCA template determines the signal, which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule, or a complex generated in a series of reactions, e.g., hybridization, ligation, extension, replication, transcription/reverse transcription, and/or amplification (e.g., rolling circle amplification), in any suitable combination.

(iii) Detection and Analysis

In some embodiments, the analyzing further comprises use of one or more detectably-labeled probe labeled with a fluorescent moiety. In some embodiments, the detectably-labeled probe is conjugated and/or covalently linked to the fluorescent moiety. In some embodiments, the fluorescent moiety is a fluorophore. In some embodiments, the analyzing comprises detecting a signal associated with the detectably-labeled probe. In some embodiments, the analyzing comprises contacting the biological sample with both the nucleic acid probe and the detectably-labeled probe simultaneously, and detecting the signal associated with the nucleic acid probe and the detectably-labeled probe. In some embodiments, the analyzing comprises contacting the biological sample with the nucleic acid probe and the detectably-labeled probe in sequential order, then detecting the signal associated with the nucleic acid probe and the detectably-labeled probe. In some embodiments, the analyzing comprises a plurality of repeated cycles of hybridization and removal of both nucleic acid probes and detectably-labeled probes simultaneously or in sequential order.

In some embodiments, the methods comprise detecting the sequence in all or a portion of a primary probe or probe set or an RCP, or detecting a sequence of the primary probe or probe set or RCP, such as one or more barcode sequences present in the primary probe or probe set or RCP. In some embodiments, the sequence of the RCP, or barcode thereof, is indicative of a sequence of the target nucleic acid to which the RCP is hybridized. In some embodiments, the analysis and/or sequence determination comprises detecting a sequence in all or a portion of the nucleic acid concatemer and/or in situ hybridization to the RCP. In some embodiments, the detection step involves sequencing by hybridization and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof).

In some embodiments, the detection or determination comprises imaging the probe (e.g., the nucleic acid probe and/or the detectably-labeled probe) hybridized to the target nucleic acid (e.g., imaging one or more nucleic acid probes and/or detectably labeled probes hybridized thereto). In some embodiments, the target nucleic acid is an mRNA in a tissue sample, and the detection or determination is performed when the target nucleic acid and/or the amplification product is in situ in the tissue sample.

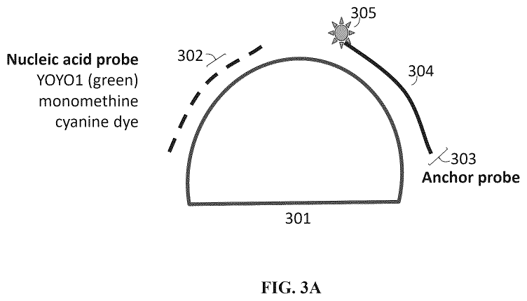

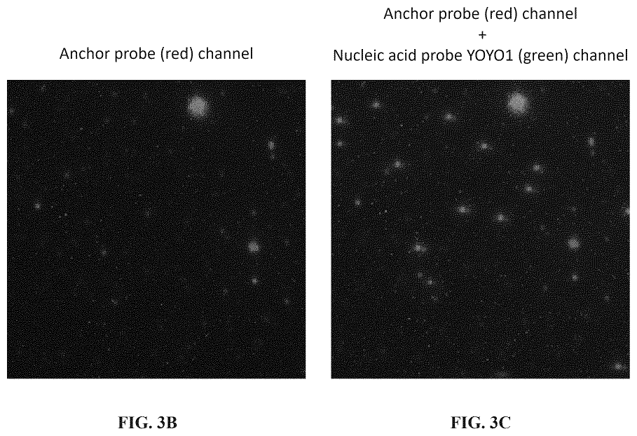

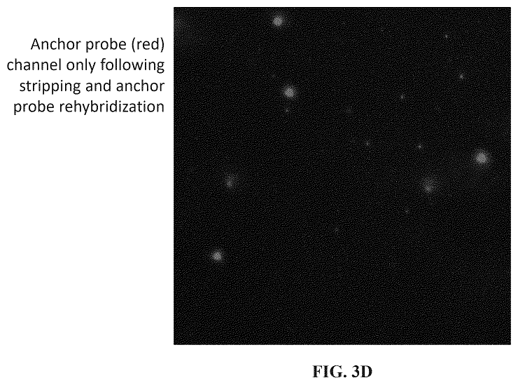

For example, FIG. 3A shows a multiplex assay wherein a single RCP (301) within a biological sample is contacted with 1) a single-stranded nucleic acid probe non-covalently associated with the green monomethine cyanine dye YOYO-1 (302) and 2) an anchor probe (303) that is covalently attached to a red fluorophore (305). Based on this combination of fluorophores, this assay is theoretically capable of multiplex detection of a single RCP using two colors. FIG. 3B shows detection of RCP using the anchor probe fluorophore (red) channel, while FIG. 3C shows detection of the RCP using the red channel and the YOYO-1-labeled nucleic acid probe (green) channel. FIG. 3D shows detection of the RCP using the anchor probe fluorophore (red) channel only following stripping of the probes from the RCP and rehybridization of the anchor probe only.

In some instances, the disclosed methods comprise the use of a branched DNA (bDNA) amplification approach to amplify signals. In branched DNA (bDNA) amplification, primary and secondary amplifier oligonucleotides, each containing multiple replicate binding sites, are assembled on, e.g., individual smFISH probes to form a branched structure which binds multiple copies of a fluorescently labeled probe (e.g., a nucleic acid probe) (Xia, et al. (2019), “Multiplexed Detection of RNA Using MERFISH and Branched DNA Amplification”, Scientific Reports 9:7721). The degree of amplification in bDNA amplification is controlled by the design of the amplification reaction, i.e., the assembled bDNA structures cannot grow indefinitely even in the presence of excess reagents, which may be used to control spot size or limit the variability in brightness from molecule to molecule (Xia, et al. (2019), ibid.).

In some instances, the disclosed methods comprise the use of a hybridization chain reaction (HCR) approach to amplify signals. In a hybridization chain reaction, two partially double-stranded nucleic acid probes self-assemble into long fluorescent polymers starting from an initiator sequence present on each probe molecule (Xia, et al. (2019), ibid.). In some embodiments, the partially double-stranded nucleic acid probes are fluorescently-labeled metastable hairpin oligonucleotides. The degree of amplification achieved through HCR can be tuned by changing the hybridization or polymerization times, and can be adjusted to achieve highly amplified signals (which may, however, increase the size of the fluorescent spots generated and/or lead to variable degrees of amplification for different copies of the same target molecule).

In some embodiments, provided herein are methods and compositions for analyzing analytes in a sample using concatemer primers and labeling agents. In various embodiments, a primer with domain on its 3′ end binds to a catalytic hairpin, and is extended with a new domain by a strand displacing polymerase. For example, a primer with domain 1 on its 3 ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer.

In various embodiments, a plurality of concatemer primers is contacted with a sample. In various embodiments, an assembly include a plurality of concatemer primers, a plurality of labeled probes, and a sample including nucleic acids. In various embodiments, each the plurality of concatemer primers each includes domain 1, 2, 3, etc. In various embodiments, each the plurality of labeled probes each include domain 1′, 2′, 3′, etc., with each corresponding domain 1′, 2′, 3′ being complementary to domain 1, 2, 3, etc., respectively. In various embodiments, the assembly includes the plurality of concatemer primers, which are capable of hybridizing to target nucleic acid sequences in the sample. Described herein is a method using the aforementioned assembly, including contacting the sample including target nucleic acids with the plurality of concatemer primers, then contacting the sample and plurality of concatemer primers with the plurality of labeled probes, thereby labeling the target nucleic acid sequences with a plurality of labeled probes. In various embodiments, the labeled probes are nucleic acid probes associated with monomethine cyanine dyes or derivatives thereof or salts thereof. See e.g., Kishi et al., SABER amplifies FISH: enhanced multiplexed imaging of RNA and DNA in cells and tissues, Nat. Methods. (2019), Saka et al., Immuno-SABER enables highly multiplexed and amplified protein imaging in tissues. Nat. Biotechnol. (2019), and U.S. Pat. Pub. No. 2021/0147902, which is fully incorporated by reference herein.

In some aspects, the provided methods comprise imaging a nucleic acid probe bound directly or indirectly to the target nucleic acid or to the primary probe or probe set or product thereof and detecting a signal associated with the monomethine cyanine dye associated with the nucleic acid probe. In some embodiments, the signal is measurable and quantifiable. In some embodiments, the nucleic acid probe further comprises a label or detectable label. In some aspects, the provided methods further comprise imaging a detectably-labeled probe bound directly or indirectly to the target nucleic acid or to the primary probe or probe set or product thereof and detecting a label or detectable label associated with the detectably-labeled probe. In some embodiments, the label and/or detectable label is measurable and quantifiable. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, the detectable label is a fluorophore that comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, cyanine-3 (Cy3), cyanine-5 (Cy5), cyanine-7 (Cy7), rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can arise from a variety of sources, including aldehyde fixation, extracellular matrix components, red blood cells, lipofuscin, and the like. Tissue background fluorescence (or autofluorescence) can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵S, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Examples of detectable nanoparticles e.g., quantum dots, comprise those described in, for example, U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. As used herein, the term “fluorescent label” comprises a signaling moiety that conveys information through the fluorescent absorption and/or emission properties of one or more molecules. Examples of fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3™-dCTP (cyanine 3-dCTP), Cy3™-dUTP (cyanine 3-dUTP), Cy5™-dCTP (cyanine 5-dCTP), Cy5™-dUTP (cyanine 5 dUTP) (Amersham Biosciences, Piscataway, N.J.), fluorescein-12-dUTP, tetramethylrhodamine-6-dUTP, TEXAS RED®-5-dUTP (red fluorescent dye-dUTP), CASCADE® BLUE-7-dUTP (blue fluorescent dye—dUTP), BODIPY™ FL-14-dUTP (green fluorescent dye-dUTP), BODIPY™ TMR-14-dUTP (orange fluorescent dye-dUTP), BODIPY™ TR-14-dUTP (red fluorescent dye-dUTP), RHODAMINE GREEN™-5-dUTP (green fluorescent dye-dUTP), OREGON GREEN™ 488-5-dUTP (green fluorescent dye-dUTP), TEXAS RED™-12-dUTP (red fluorescent dye-dUTP), BODIPY™ 630/650-14-dUTP (far red fluorescent dye-dUTP), BODIPY™ 650/665-14-dUTP (far red fluorescent dye-dUTP), ALEXA FLUOR™ 488-5-dUTP (green fluorescent dye-dUTP), ALEXA FLUOR™ 532-5-dUTP (yellow fluorescent dye-dUTP), ALEXA FLUOR™ 568-5-dUTP (red/orange fluorescent dye-dUTP), ALEXA FLUOR™ 594-5-dUTP (red fluorescent dye-dUTP), ALEXA FLUOR™ 546-14-dUTP (orange fluorescent dye-dUTP), fluorescein-12-UTP, tetramethylrhodamine-6-UTP, TEXAS RED™-5-UTP (red fluorescent dye-UTP), mCherry, CASCADE® BLUE-7-UTP (blue fluorescent dye-UTP), BODIPY™ FL-14-UTP (green fluorescent protein-UTP), BODIPY™ TMR-14-UTP (orange fluorescent dye-UTP), BODIPY™ TR-14-UTP (red fluorescent dye-UTP), RHODAMINE GREEN™-5-UTP (green fluorescent dye-UTP), ALEXA FLUOR™ 488-5-UTP (green fluorescent dye-UTP), and ALEXA FLUOR™ 546-14-UTP (orange fluorescent dye-UTP) (Molecular Probes, Inc. Eugene, Oreg.). Methods are known for custom synthesis of nucleotides having other fluorophores.

Other fluorophores available for post-synthetic attachment comprise, but are not limited to, ALEXA FLUOR™ dyes (fluorescent dyes) such as ALEXA FLUOR™ 350 (blue fluorescent dye), ALEXA FLUOR™ 594 (red fluorescent dye), and ALEXA FLUOR™ 647 (far red fluorescent dye); BODIPY™ dyes (fluorescent dyes) such as BODIPY™ FL (green fluorescent dye), BODIPY™ TMR (orange fluorescent dye), and BODIPY™ 650/665 (far red fluorescent dye); Cascade® Blue (blue fluorescent dye), Cascade® Yellow (yellow fluorescent dye), Dansyl, lissamine rhodamine B, Marina Blue™ (blue fluorescent dye), Oregon Green™ 488, Oregon Green™ 514, Pacific Blue, rhodamine 6G, rhodamine green, rhodamine red, tetramethyl rhodamine, Texas Red® (red fluorescent dye) (available from Molecular Probes, Inc., Eugene, Oreg.), Cy2™ (cyanine 2), Cy3.5™ (cyanine 3.5), Cy5.5™ (cyanine 5.5), and Cy7™ (cyanine 7) (Amersham Biosciences, Piscataway, N.J.). FRET tandem fluorophores may also be used, comprising, but not limited to, PerCP-Cy™5.5 (far red fluorescent tandem fluorophore), PE-Cy™5 (red fluorescent tandem fluorophore), PE-Cy™5.5 (red fluorescent tandem fluorophore), PE-Cy™7 (far red fluorescent tandem fluorophore), PE-Texas Red® (red fluorescent tandem fluorophore), APC-Cy™7 (far red fluorescent tandem fluorophore), PE-Alexa™ dyes (e.g., 610, 647, 680), and APC-Alexa™ dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence is indirectly labeled, especially with a hapten that is then bound by a capture agent, e.g., as disclosed in U.S. Pat. Nos. 5,344,757, 5,702,888, and 5,198,537, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a simpler set up like an epifluorescence microscope, or a more complicated design such as a confocal microscope, which uses optical sectioning to get better resolution of the fluorescent image.

In some embodiments, confocal microscopy is used for detection and imaging of a probe (e.g., the nucleic acid probe and/or the detectably-labeled probe). Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, detection of the barcode sequences is performed by sequential hybridization of probes (e.g., nucleic acid probes and/or detectably-labeled probes) to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and a universal pool of nucleic acid probes. In some cases, the sequences of signal codes comprise fluorophore sequences assigned to the corresponding barcode sequences or complements thereof. In some embodiments, the nucleic acid probes are labeled with monomethine cyanine dyes or derivatives thereof or salts thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In some embodiments, the analyzing step comprises contacting the biological sample with one or more nucleic acid probes that directly or indirectly hybridize to the barcode sequences or complements thereof (e.g., in amplification products generated using the probes or probe sets), detecting a signal associated with a monomethine cyanine dye associated with the one or more nucleic acid probes and dehybridizing the one or more nucleic acid probes. In some embodiments, the contacting and dehybridizing steps are repeated with one or more detectably labeled probes and/or one or more other nucleic acid probes that directly or indirectly hybridize to the barcode sequences or complements thereof. In some aspects, the method comprises sequential hybridization of nucleic acid probes to create a spatiotemporal signal signature or code that identifies the analyte. In some embodiments, the method further comprises sequential hybridization of nucleic acid probes and detectably-labeled probes to create a spatiotemporal signal signature or code that identified the analyte.

In any of the embodiments herein, the method step comprises contacting the biological sample with one or more first nucleic acid probes that directly hybridize to the plurality of probes or probe sets. In some instances, the method comprises contacting the biological sample with one or more first nucleic acid probes that indirectly hybridize to the plurality of probes or probe sets. In any of the embodiments herein, the method comprises contacting the biological sample with one or more first nucleic acid probes that directly or indirectly hybridize to the plurality of probes or probe sets.

In any of the embodiments herein, the method comprises contacting the biological sample with one or more intermediate probes that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more nucleic acid probes. In any of the embodiments herein, the detecting step further comprises dehybridizing the one or more intermediate probes and/or the one or more nucleic acid probes from the barcode sequences or complements thereof (e.g., of the plurality of probes or probe sets or rolling circle amplification product generated using the plurality of probes or probe sets). In some embodiments, the contacting and dehybridizing steps are repeated with the one or more intermediate probes, the one or more nucleic acid probes, one or more other intermediate probes, and/or one or more other nucleic acid probes. In some embodiments, the contacting and dehybridizing steps are further repeated with the one or more intermediate probes and one or more detectably-labeled probes. In some cases, the repeated contacting, detection and dehybridizing steps allows detection of barcode sequences or complements thereof and identification of the corresponding sequences of signal codes (e.g., fluorophore sequences assigned to the corresponding barcode sequences or complements thereof).

In some embodiments, nucleic acid hybridization is used for sequencing. These methods utilize labeled nucleic acid decoder probes that are complementary to at least a portion of a barcode sequence. Multiplex decoding can be performed with pools of many different probes with distinguishable labels. In some embodiments, the labeled nucleic acid decoder probes are nucleic acid probes associated with monomethine cyanine dyes or derivatives thereof or salts thereof. A non-limiting examples of nucleic acid hybridization sequencing is described for example in U.S. Pat. No. 8,460,865, which is herein incorporated by reference in its entirety.

In some embodiments, real-time monitoring of DNA polymerase activity is used during sequencing. For example, nucleotide incorporations can be detected through fluorescence resonance energy transfer (FRET), as described for example in Korlach et al., Proc. Natl. Acad. Sci. USA (2008), 105, 1176-1181.

In some embodiments, the analysis and/or sequence determination involves washing to remove unbound polynucleotides, thereafter, revealing a fluorescent product for imaging.

### III. Samples, Analytes, and Target Sequences

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein can be any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular probe or circularizable probe or probe set. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Any suitable method for sample permeabilization can generally be used in connection with the samples described herein.

In some embodiments, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample can be embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. No. 6,391,937 and materials for sample expansion as described, for example, in U.S. Patent Application Publication Nos. 2017/0253918 and 2018/0052081, all of which are herein incorporated by reference in their entireties.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, the contents of which are incorporated herein by reference its entirety.

### B. Analytes

A biological sample may comprise one or a plurality of analytes of interest. Methods for performing multiplexed assays to analyze two or more different analytes in a single biological sample are provided. The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of an RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus, in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and includes nucleic acid analytes and non-nucleic acid analytes. In some embodiments, the analyte is a target nucleic acid. Methods and compositions disclosed herein can be used to analyze nucleic acid analytes (e.g., using a probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. In some embodiments, the RNA comprises circular RNA. In some embodiments, the RNA is a bacterial rRNA (e.g., 16s rRNA or 23s rRNA).

In some embodiments described herein, an analyte is a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent includes an agent that interacts with an analyte (e.g., an endogenous analyte in a sample). In some embodiments, the labeling agents comprises a reporter oligonucleotide that is indicative of the analyte or portion thereof interacting with the labeling agent. For example, the reporter oligonucleotide may comprise a barcode sequence that permits identification of the labeling agent. In some embodiments, the reporter oligonucleotide is the target nucleic acid targeted by the preceding methods. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the probe is a nucleic acid probe. In some embodiments, the probe is a circular probe or a circularizable probe or probe set, and a nucleic acid probe hybridizes to a circular template, a complement thereof, or a product thereof derived from the circular probe or the circularizable probe or probe set.

In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety includes any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. Protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent comprises a reporter oligonucleotide and a label. A label can be fluorophore, a radioisotope, a molecule capable of a colorimetric reaction, a magnetic particle, or any other suitable molecule or compound capable of detection. In some embodiments, the label is conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample are subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### C. Barcode Sequences

In some embodiments, an analyte described herein is associated with one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### IV. Compositions and Kits

In some aspects, provided herein are compositions comprising any of the nucleic acid probes described herein. Also provided herein are kits, for analyzing an analyte in a biological sample according to any of the methods described herein. In some embodiments, provided herein is a kit comprising a nucleic acid probe non-covalently associated with a monomethine cyanine dye or salt thereof. The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some embodiments, the kits comprise a polymerase suitable for a gapfill reaction. In some embodiments, the polymerase suitable for a gapfill reaction lacks strand-displacement activity. Example polymerases that lack strand-displacement activity include, but are not limited to, T4 DNA polymerase and T7 DNA polymerase. In some embodiments, the kits comprise a polymerase suitable for rolling circle amplification. In some embodiments, the polymerase suitable for rolling circle amplification has strand-displacement activity. Example polymerases with strand-displacement activity include, but are not limited to, Bst and Phi29. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example, nucleic acid primers.

### V. Opto-Fluidic Instruments for Analysis of Biological Samples

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., as described in Section IV). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

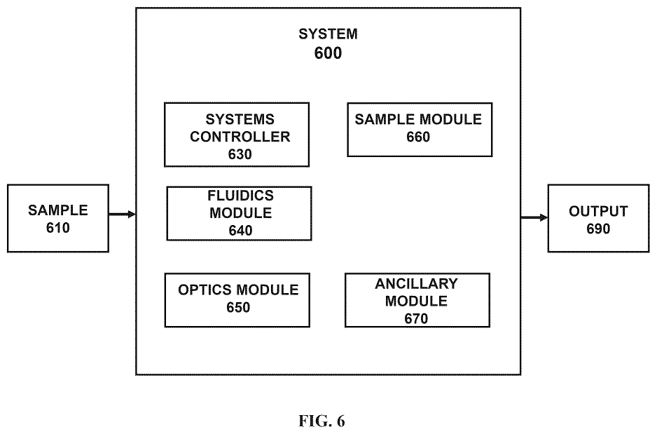

FIG. 6 shows an example workflow of analysis of a biological sample 610 (e.g., cell or tissue sample) using an opto-fluidic instrument 600, according to various embodiments. In various embodiments, the sample 610 comprises a biological sample (e.g., a tissue) that includes molecules such as DNA, RNA, proteins, antibodies, etc. For example, the sample 610 can be a sectioned tissue that is treated to access the RNA thereof for labeling with probes described herein. Ligation of the probes may generate a circular probe which can be enzymatically amplified and bound with detectably labeled probes, which can create bright signal that is convenient to image and has a high signal-to-noise ratio.

In various embodiments, the sample 610 may be placed in the opto-fluidic instrument 600 for analysis and detection of the molecules in the sample 610. In various embodiments, the opto-fluidic instrument 600 can be a system configured to facilitate the experimental conditions conducive for the detection of the target molecules. For example, the opto-fluidic instrument 600 can include a fluidics module 640, an optics module 650, a sample module 660, and an ancillary module 670, and these modules may be operated by a system controller 630 to create the experimental conditions for the probing of the molecules in the sample 610 by selected probes (e.g., circularizable DNA probes), as well as to facilitate the imaging of the probed sample (e.g., by an imaging system of the optics module 650). In various embodiments, the various modules of the opto-fluidic instrument 600 may be separate components in communication with each other, or at least some of them may be integrated together.

In various embodiments, the sample module 660 may be configured to receive the sample 610 into the opto-fluidic instrument 600. For instance, the sample module 660 may include a sample interface module (SIM) that is configured to receive a sample device (e.g., cassette) onto which the sample 610 can be deposited. That is, the sample 610 may be placed in the opto-fluidic instrument 600 by depositing the sample 610 (e.g., the sectioned tissue) on a sample device that is then inserted into the SIM of the sample module 660. In some instances, the sample module 660 may also include an X-Y stage onto which the SIM is mounted. The X-Y stage may be configured to move the SIM mounted thereon (e.g., and as such the sample device containing the sample 610 inserted therein) in perpendicular directions along the two-dimensional (2D) plane of the opto-fluidic instrument 600.

The experimental conditions that are conducive for the detection of the molecules in the sample 610 may depend on the target molecule detection technique that is employed by the opto-fluidic instrument 600. For example, in various embodiments, the opto-fluidic instrument 600 can be a system that is configured to detect molecules in the sample 610 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 610 using reagents such as washing/stripping reagents, hybridizing reagents, etc., and such reagents may be provided by the fluidics module 640.

In various embodiments, the fluidics module 640 may include one or more components that may be used for storing the reagents, as well as for transporting said reagents to and from the sample device containing the sample 610. For example, the fluidics module 640 may include reservoirs configured to store the reagents, as well as a waste container configured for collecting the reagents (e.g., and other waste) after use by the opto-fluidic instrument 600 to analyze and detect the molecules of the sample 610. Further, the fluidics module 640 may also include pumps, tubes, pipettes, etc., that are configured to facilitate the transport of the reagent to the sample device (e.g., and as such the sample 610). For instance, the fluidics module 640 may include pumps (“reagent pumps”) that are configured to pump washing/stripping reagents to the sample device for use in washing/stripping the sample 610 (e.g., as well as other washing functions such as washing an objective lens of the imaging system of the optics module 650).

In various embodiments, the ancillary module 670 can be a cooling system of the opto-fluidic instrument 600, and the cooling system may include a network of coolant-carrying tubes that are configured to transport coolants to various modules of the opto-fluidic instrument 600 for regulating the temperatures thereof. In such cases, the fluidics module 640 may include coolant reservoirs for storing the coolants and pumps (e.g., “coolant pumps”) for generating a pressure differential, thereby forcing the coolants to flow from the reservoirs to the various modules of the opto-fluidic instrument 600 via the coolant-carrying tubes. In some instances, the fluidics module 640 may include returning coolant reservoirs that may be configured to receive and store returning coolants, i.e., heated coolants flowing back into the returning coolant reservoirs after absorbing heat discharged by the various modules of the opto-fluidic instrument 600. In such cases, the fluidics module 640 may also include cooling fans that are configured to force air (e.g., cool and/or ambient air) into the returning coolant reservoirs to cool the heated coolants stored therein. In some instance, the fluidics module 640 may also include cooling fans that are configured to force air directly into a component of the opto-fluidic instrument 600 so as to cool said component. For example, the fluidics module 640 may include cooling fans that are configured to direct cool or ambient air into the system controller 630 to cool the same.

As discussed above, the opto-fluidic instrument 600 may include an optics module 650 which include the various optical components of the opto-fluidic instrument 600, such as but not limited to a camera, an illumination module (e.g., LEDs), an objective lens, and/or the like. The optics module 650 may include a fluorescence imaging system that is configured to image the fluorescence emitted by the probes (e.g., oligonucleotides) in the sample 610 after the probes are excited by light from the illumination module of the optics module 650.

In some instances, the optics module 650 may also include an optical frame onto which the camera, the illumination module, and/or the X-Y stage of the sample module 660 may be mounted.

In various embodiments, the system controller 630 may be configured to control the operations of the opto-fluidic instrument 600 (e.g., and the operations of one or more modules thereof). In some instances, the system controller 630 may take various forms, including a processor, a single computer (or computer system), or multiple computers in communication with each other. In various embodiments, the system controller 630 may be communicatively coupled with data storage, set of input devices, display system, or a combination thereof. In some cases, some or all of these components may be considered to be part of or otherwise integrated with the system controller 630, may be separate components in communication with each other, or may be integrated together. In other examples, the system controller 630 can be, or may be in communication with, a cloud computing platform.

In various embodiments, the opto-fluidic instrument 600 may analyze the sample 610 and may generate the output 690 that includes indications of the presence of the target molecules in the sample 610. For instance, with respect to the example embodiment discussed above where the opto-fluidic instrument 600 employs a hybridization technique for detecting molecules, the opto-fluidic instrument 600 may cause the sample 610 to undergo successive rounds of detectably labeled probe hybridization (e.g., using two or more sets of fluorescent probes, where each set of fluorescent probes is excited by a different color channel) and be imaged to detect target molecules in the probed sample 610. In such cases, the output 690 may include optical signatures (e.g., a codeword) specific to each gene, which allow the identification of the target molecules.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### Exemplary Embodiments

The following embodiments are exemplary and are not intended to limit the scope of the invention as claimed.

Embodiment 1. A method, comprising: (a) contacting a biological sample comprising a target nucleic acid, or an extension or amplification product thereof, with a nucleic acid probe and a monomethine cyanine dye, or a salt thereof, wherein the nucleic acid probe is non-covalently associated with the monomethine cyanine dye, or a salt thereof, thereby hybridizing the nucleic acid probe to the target nucleic acid or the extension or amplification product thereof at a location in the biological sample; and (b) detecting, at the location, a signal associated with the monomethine cyanine dye or a salt thereof.

Embodiment 2. The method of embodiment 1, wherein the monomethine cyanine dye, or a salt thereof, is selected from the group consisting of a thioalkyl derivative, 2-iminobenzo-thiazoline, a sulfobetaine salt of N-alkyl heterocycle, 2-chloroquinoline, 4-chloroquinoline, 3-phenyl-2H-1,4-benzothiazine, indole-3-carboxaldehyde, methyl-pyridinium iodide, thiazole orange or a derivative thereof, oxazole yellow or a derivative thereof, and oxazole blue or a derivative thereof.

Embodiment 3. The method of embodiment 1, wherein the nucleic acid probe is in a single-stranded form prior to the hybridizing.

Embodiment 4. The method of embodiment 1, wherein the monomethine cyanine dye is a monomeric monomethine cyanine dye.

Embodiment 5. The method of embodiment 4, wherein the monomeric monomethine cyanine dye has the structure:

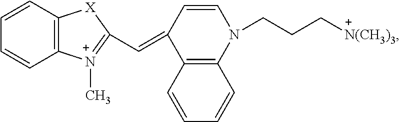

wherein X is O or S.

Embodiment 6. The method of embodiment 4, wherein the monomeric monomethine cyanine dye has the structure:

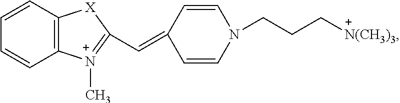

wherein X is O or S.

Embodiment 7. The method of embodiment 4, wherein the monomeric monomethine cyanine dye has the structure:

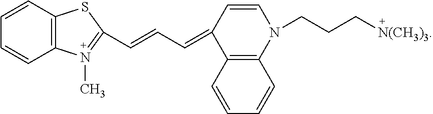

Embodiment 8. The method of embodiment 1, wherein the nucleic acid probe is in a partially double-stranded form prior to the hybridizing.

Embodiment 9. The method of embodiment 8, wherein the partially double-stranded form comprises a hairpin form.

Embodiment 10. The method of embodiment 1, wherein the monomethine cyanine dye is a dimeric monomethine cyanine dye.

Embodiment 11. The method of embodiment 10, wherein the dimeric monomethine cyanine dye has the structure:

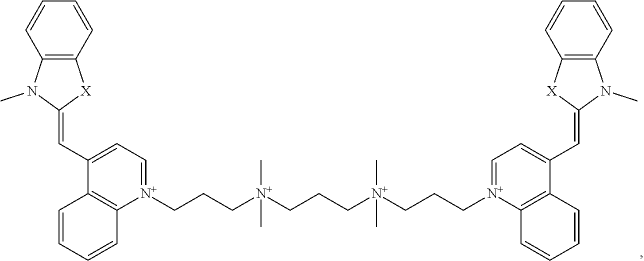

wherein X is S or O.

Embodiment 12. The method of embodiment 10, wherein the dimeric monomethine cyanine dye has the structure:

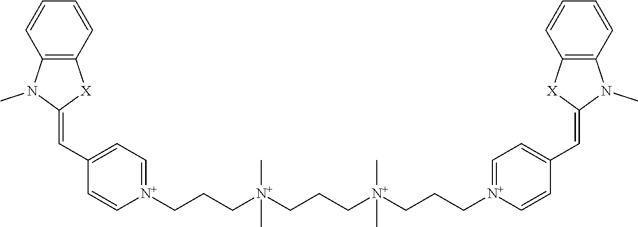

wherein X is S or O.

Embodiment 13. The method of embodiment 10, wherein the dimeric monomethine cyanine dye has the structure:

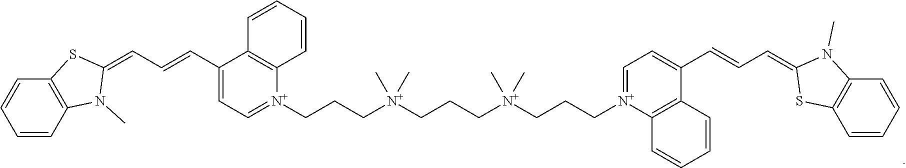 [SMILES: CN1/C(=C/C=C/c2cc[n+](CCC[N+](C)(C)CCC[N+](C)(C)CCC[n+]3ccc(/C=C/C=C4/Sc5ccccc5N4C)c4ccccc43)c3ccccc23)Sc2ccccc21]

Embodiment 14. The method of embodiment 1, further comprising, prior to the contacting, incubating the nucleic acid probe in a solution comprising the monomethine cyanine dye, or a salt thereof, thereby non-covalently associating the nucleic acid probe with the monomethine cyanine dye, or a salt thereof.

Embodiment 15. The method of embodiment 1, wherein the contacting is for a duration of about 30 seconds to about 2 minutes.

Embodiment 16. The method of embodiment 1, wherein the contacting comprises hybridizing the nucleic acid probe to a rolling circle amplification product (RCP) associated with a target nucleic acid at the location in the biological sample.

Embodiment 17. The method of embodiment 16, wherein the RCP is generated from a circular template at the location in the biological sample.

Embodiment 18. The method of embodiment 17, further comprising: contacting the biological sample with a circularizable probe or probe set that directly or indirectly binds to the target nucleic acid at the location in the biological sample, and circularizing the circularizable probe or probe set to generate the circular template.

Embodiment 19. The method of embodiment 1, wherein the detecting comprises detecting a fluorescence signal of the monomethine cyanine dye, or a salt thereof, using fluorescence-based microscopy.

Embodiment 20. A method, comprising: (a) contacting a biological sample comprising an amplicon of a target nucleic acid molecule with (i) a nucleic acid probe comprising a single stranded region and a double-stranded region, and (ii) a double-intercalating monomethine cyanine dye, or a salt thereof, that is non-covalently associated with the double-stranded region, thereby hybridizing the nucleic acid probe to the amplicon of the target nucleic acid molecule at a location in the biological sample; and (b) detecting, at the location, a signal associated with the monomethine cyanine dye, or a salt thereof.

Embodiment 21. A system for detecting a target nucleic acid, the system comprising: a) a biological sample comprising a target nucleic acid or an extension or amplification product thereof, and b) a nucleic acid probe non-covalently associated with a monomethine cyanine dye, or a salt thereof, wherein the nucleic acid probe is hybridized to the target nucleic acid or the extension or amplification product thereof.

Embodiment 22. A kit comprising: (a) a circularizable probe or probe set that is complementary to a target nucleic acid of a biological sample; and (b) a nucleic acid probe non-covalently associated with a monomethine cyanine dye, or a salt thereof, wherein the nucleic acid probe comprises a sequence complementary to an extension or amplification product of the target nucleic acid.

Embodiment 23. A method, comprising: (a) contacting a biological sample with a nucleic acid probe, wherein the nucleic acid probe is non-covalently associated with a monomethine cyanine dye or a salt thereof, wherein the contacting results in the nucleic acid probe binding directly or indirectly to a target nucleic acid at a location in the biological sample; and (b) detecting a signal associated with the monomethine cyanine dye or the salt thereof.

Embodiment 24. A method, comprising: (a) contacting a biological sample with a nucleic acid probe, wherein the nucleic acid probe is non-covalently associated with a monomethine cyanine dye or a salt thereof, wherein the contacting results in the nucleic acid probe binding to a target nucleic acid or an extension/amplification product thereof at a location in the biological sample; and (b) detecting a signal associated with the monomethine cyanine dye or the salt thereof.

Embodiment 25. The method of embodiment 23 or claim 2, wherein the monomethine cyanine dye or the salt thereof comprises and/or is derived from a thioalkyl derivative, 2-iminobenzo-thiazoline, a sulfobetaine salt of N-alkyl heterocycle, 2-chloroquinoline, 4-chloroquinoline, 3-phenyl-2H-1,4-benzothiazine, indole-3-carboxaldehyde, methyl-pyridinium iodide, thiazole orange or a derivative thereof, oxazole yellow or a derivative thereof, or oxazole blue or a derivative thereof.

Embodiment 26. The method of embodiment 25, wherein the monomethine cyanine dye or the salt thereof is derived from thiazole orange, oxazole yellow, or oxazole blue.

Embodiment 27. The method of any one of embodiments 23-26, wherein the monomethine cyanine dye or the salt thereof is monomeric.

Embodiment 28. The method of any one of embodiments 23-27, wherein the monomethine cyanine dye or the salt thereof is dimeric.

Embodiment 29. A method, comprising: a) providing a nucleic acid probe that is non-covalently associated with a monomethine cyanine dye or salt thereof, b) contacting a biological sample with the nucleic acid probe, wherein the contacting results in the nucleic acid probe binding directly or indirectly to a target nucleic acid at a location in the biological sample; and c) detecting a signal associated with the monomethine cyanine dye or the salt thereof.

Embodiment 30. A method, comprising: a) providing a nucleic acid probe that is non-covalently associated with a monomethine cyanine dye or salt thereof, b) contacting a biological sample with the nucleic acid probe, wherein the contacting results in the nucleic acid probe binding to a target nucleic acid or extension/amplification product thereof at a location in the biological sample; and c) detecting a signal associated with the monomethine cyanine dye or the salt thereof.

Embodiment 31. The method of embodiment 29 or claim 8, wherein the nucleic acid probe is provided in a single-stranded form.

Embodiment 32. The method of any one of embodiments 29-31, wherein the monomethine cyanine dye is a monomeric monomethine cyanine dye.

Embodiment 33. The method of embodiment 32, wherein the monomeric monomethine cyanine dye has the structure:

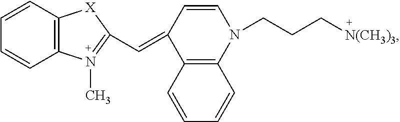

wherein X is O or S.

Embodiment 34. The method of embodiment 32, wherein the monomeric monomethine cyanine dye has the structure:

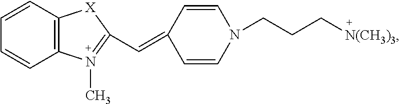

wherein X is O or S.

Embodiment 35. The method of embodiment 32, wherein the monomeric monomethine cyanine dye has the structure:

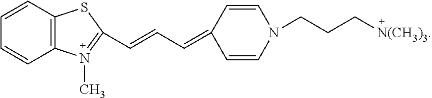

Embodiment 36. The method of embodiment 29 or claim 8, wherein the nucleic acid probe is provided in partially double-stranded form.

Embodiment 37. The method of embodiment 36, wherein the partially double-stranded form comprises a hairpin form.

Embodiment 38. The method of embodiment 36 of claim 15, wherein the monomethine cyanine dye is a dimeric monomethine cyanine dye.

Embodiment 39. The method of embodiment 38, wherein the dimeric monomethine cyanine dye has a structure of formula:

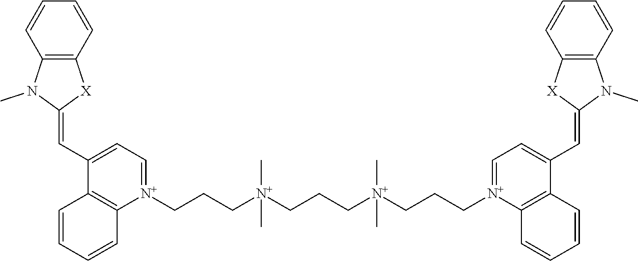

wherein X is S or O.

Embodiment 40. The method of embodiment 38, wherein the dimeric monomethine cyanine dye has a structure of formula:

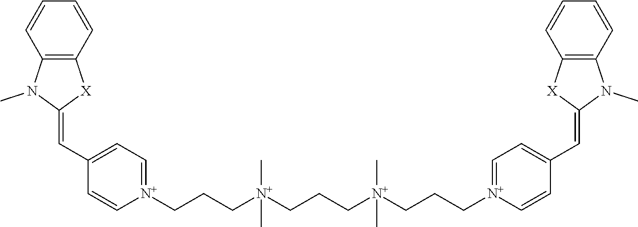

wherein X is S or 0.

Embodiment 41. The method of embodiment 38, wherein the dimeric monomethine cyanine dye has the structure:

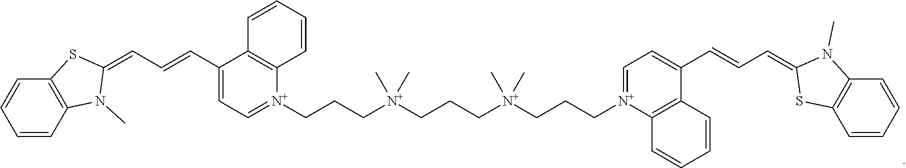 [SMILES: CN1/C(=C/C=C/c2cc[n+](CCC[N+](C)(C)CCC[N+](C)(C)CCC[n+]3ccc(/C=C/C=C4/Sc5ccccc5N4C)c4ccccc43)c3ccccc23)Sc2ccccc21]

Embodiment 42. The method of any one of embodiments 23-41, wherein the nucleic acid probe is RNA.

Embodiment 43. The method of any one of embodiments 23-41, wherein the nucleic acid probe is DNA.

Embodiment 44. The method of any one of embodiments 29-43, wherein providing the nucleic acid probe with the monomethine cyanine dye or the salt thereof comprises incubating a nucleic acid molecule in a solution comprising the monomethine cyanine dye or the salt thereof.

Embodiment 45. The method of embodiment 23 or claim 2, wherein the contacting is for a duration of about 30 seconds to about 2 minutes.

Embodiment 46. The method of embodiment 44 or claim 23, wherein the solution is an aqueous solution.

Embodiment 47. The method of embodiment 44 or claim 23, wherein the solution is an organic solution.

Embodiment 48. The method of any one of embodiments 23-47, wherein the nucleic acid probe hybridizes to a rolling circle amplification product (RCP) associated with the target nucleic acid at the location in the biological sample.

Embodiment 49. The method of embodiment 48, wherein the RCP is generated from a circular template at the location in the biological sample.

Embodiment 50. The method of embodiment 49, comprising contacting the biological sample with the circular template.

Embodiment 51. The method of embodiment 50, wherein the circular template is a circular probe that directly or indirectly binds to the target nucleic acid at the location in the biological sample.

Embodiment 52. The method of embodiment 49, comprising generating the circular template at the location in the biological sample.

Embodiment 53. The method of embodiment 52, comprising reverse transcribing an RNA in the biological sample to generate a molecule comprising a cDNA of the RNA and circularizing the molecule comprising the cDNA to generate the circular template.

Embodiment 54. The method of embodiment 52, comprising contacting the biological sample with a circularizable probe or probe set that directly or indirectly binds to the target nucleic acid at the location in the biological sample, and circularizing the circularizable probe or probe set to generate the circular template.

Embodiment 55. The method of embodiment 54, wherein the circularizable probe or probe set hybridizes to the target nucleic acid, and the circularizable probe or probe set is circularized using the target nucleic acid as a template.

Embodiment 56. The method of embodiment 55, wherein hybridization of the circularizable probe or probe set to the target nucleic acid forms a gap between the ends of the circularizable probe or probe set, and wherein generating the circular template comprises filling the gap between the ends of the circularizable probe or probe set.

Embodiment 57. The method of any one of embodiments 49-56, wherein the circular template does not comprise a barcode sequence assigned to associate with the target nucleic acid or a sequence thereof.

Embodiment 58. The method of any one of embodiments 49-56, wherein the circular template comprises a barcode sequence assigned to associate with the target nucleic acid or a sequence thereof.

Embodiment 59. The method of any one of embodiments 23-58, wherein the target nucleic acid is a cellular nucleic acid in the biological sample.

Embodiment 60. The method of embodiment 59, wherein the cellular nucleic acid is genomic DNA.

Embodiment 61. The method of embodiment 59, wherein the cellular nucleic acid is RNA.

Embodiment 62. The method of embodiment 61, wherein the RNA is mRNA, miRNA, lnRNA, or rRNA.

Embodiment 63. The method of embodiment 62, wherein the RNA is mRNA.

Embodiment 64. The method of any one of embodiments 23-58, wherein the target nucleic acid is a reporter oligonucleotide comprised on a labeling agent, wherein the labeling agent is bound to an analyte in the biological sample.

Embodiment 65. The method of embodiment 64, wherein the analyte is a nucleic acid analyte.

Embodiment 66. The method of embodiment 64, wherein the analyte is a polypeptide.

Embodiment 67. The method of any one of embodiments 23-66, wherein the signal associated with the monomethine cyanine dye or the salt thereof is a fluorescence signal.

Embodiment 68. The method of embodiment 67, comprising detecting the fluorescence signal using fluorescence-based microscopy.

Embodiment 69. The method of any one of embodiments 23-68, wherein the biological sample is a fresh tissue sample, a frozen tissue sample, or a fixed tissue sample.

Embodiment 70. The method of any one of embodiments 23-69, wherein the biological sample is a fresh frozen tissue section or a formalin-fixed paraffin-embedded tissue section.

Embodiment 71. The method of any one of embodiments 23-70, wherein the biological sample is derived from a human.

Embodiment 72. The method of any one of embodiments 23-71, wherein the biological sample is derived from a human with a disease or condition.

Embodiment 73. The method of any one of embodiments 23-70, wherein the biological sample is derived from a non-human mammal.

Embodiment 74. A method, comprising: a) contacting a biological sample with a nucleic acid probe, wherein the nucleic acid probe is non-covalently associated with a monomethine cyanine dye or a salt thereof, wherein the nucleic acid probe hybridizes to a region of a rolling circle amplification product (RCP) at a location in the biological sample, wherein the RCP is associated with a target nucleic acid in the biological sample; and b) detecting a signal associated with the monomethine cyanine dye or salt thereof, thereby detecting the target nucleic acid in the biological sample.

Embodiment 75. The method of embodiment 74, wherein the monomethine cyanine dye or the salt thereof comprises and/or is derived from a thioalkyl derivate, 2-iminobenzo-thiazoline, a sulfobetaine salt of N-alkyl heterocycle, 2-chloroquinoline, 4-chloroquinoline, 3-phenyl-2H-1,4-benzothiazine, indole-3-carboxaldehyde, methyl-pyridinium iodide, thiazole orange or a derivative thereof, oxazole yellow or a derivative thereof, or oxazole blue or a derivative thereof.

Embodiment 76. The method of embodiment 74 or claim 53, wherein the monomethine cyanine dye or the salt thereof is monomeric.

Embodiment 77. The method of embodiment 74 or claim 53, wherein the monomethine cyanine dye or the salt thereof is dimeric.

Embodiment 78. A method, comprising: a) providing a nucleic acid probe non-covalently associated with a monomethine cyanine dye; b) contacting a biological sample with the nucleic acid probe, wherein the nucleic acid probe hybridizes to a region of a rolling circle amplification product (RCP) at a location in the biological sample, wherein the RCP is associated with a target nucleic acid in the biological sample; and c) detecting a signal associated with the monomethine cyanine dye or salt thereof, thereby detecting the target nucleic acid in the biological sample.

Embodiment 79. The method of embodiment 78, wherein the nucleic acid probe is provided in a single-stranded form.

Embodiment 80. The method of embodiment 79, wherein the monomethine cyanine dye is a monomeric monomethine cyanine dye.

Embodiment 81. The method of embodiment 78, wherein the nucleic acid probe is provided in a partially double-stranded form.

Embodiment 82. The method of embodiment 81, wherein the monomethine cyanine dye is a dimeric monomethine cyanine dye.

Embodiment 83. A method, comprising: a) contacting a biological sample with a nucleic acid probe, wherein the nucleic acid probe comprises a hairpin form, wherein the nucleic acid probe is non-covalently associated with a double-intercalating monomethine cyanine dye or a salt thereof, wherein the nucleic acid probe hybridizes to a region of a rolling circle amplification product (RCP), wherein the RCP is associated with a target nucleic acid at a location in the biological sample; and b) detecting a signal associated with the monomethine cyanine dye or salt thereof.

Embodiment 84. A system for detecting a target nucleic acid, the system comprising: a) a biological sample comprising a target nucleic acid or an extension or amplification product thereof, and b) a nucleic acid probe non-covalently associated with a monomethine cyanine dye or a salt thereof, wherein the nucleic acid probe is hybridized to the target nucleic acid or the extension or amplification product thereof.

Embodiment 85. The system of embodiment 84, wherein the nucleic acid probe is hybridized to the extension or amplification product thereof.

Embodiment 86. The system of embodiment 84 or claim 63, wherein the extension or amplification product is a rolling circle amplification product.

Embodiment 87. The system of embodiment 86, wherein the biological sample is on a slide or in a well.

Embodiment 88. The system of any one of embodiments 84-87, wherein the system further comprises an imaging device, and wherein the slide or the well is on the imaging device.

Embodiment 89. A kit comprising: (a) a circularizable probe or probe set that is complementary to a target nucleic acid of a biological sample; and (b) a nucleic acid probe non-covalently associated with a monomethine cyanine dye or a salt thereof, wherein the nucleic acid probe comprises a sequence complementary to an extension or amplification product of the target nucleic acid.

Embodiment 90. The kit of embodiment 89, further comprising a ligase for forming a circular template from the circularizable probe or probe set.

Embodiment 91. The kit of embodiment 89 or claim 68, further comprising a polymerase suitable for a gapfill reaction.

Embodiment 92. The kit of any one of embodiments 89-91, further comprising a mixture of free nucleotides including the four canonical bases: adenine, thymine, guanine, and cytosine.

Embodiment 93. The kit of any one of embodiments 89-92, further comprising a polymerase suitable for rolling circle amplification.

Embodiment 94. The kit of any one of embodiments 89-93, wherein the circularizable probe comprises a primer binding sequence, and the kit further comprising a primer.

### EXAMPLES

The following examples are included for illustrative purposes only and are not intended to limit the scope of the present disclosure.

### Example 1: In Situ Detection of a Target Nucleic Acid Using a Nucleic Acid Probe Associated with a Monomethine Cyanine Dye

This example describes use of a single-stranded nucleic acid probe associated with a monomethine cyanine dye (MCD) in an in situ model system for detection of a target nucleic acid at a location within a sample.

Copies of a synthetic nucleic acid encoding PLR2A (DNA-directed RNA polymerase II subunit RPB1), referred to as a “pseudogene” here, were embedded in a hydrogel on a slide to generate an in situ model system. To prepare for probe hybridization, a wash buffer was added. Circularizable probes targeting RNA of the pseudogene were hybridized to the target and ligated to form circular templates for RCA. The circularizable probes included a first end sequence and a second end sequence each complementary to two adjacent sequences in the pseudogene. The circularizable probes also included a first detection sequence and a second detection sequence. The sample was then incubated with an RCA mixture containing a strand-displacing DNA polymerase and dNTPs for RCA of the circularized probes to form an RCA product (“RCP”).

Next, an MCD-stained probe specific for the first detection sequence was generated. A single stranded DNA (1 ml of 100 M) was incubated in a standard nucleic acid hybridization buffer with 1 μL of 1 mM YOYO1 dye in DMSO for 1 hour at room temperature. To prepare the anchor probe, an anchor nucleic acid molecule was covalently linked to a 647N fluorophore through standard methods. The anchor probe was complementary to a second target sequence of the RCP. A schematic of the relationship between the nucleic acid probe, the anchor probe, and the RCP is shown in FIG. 3A.

The sample was then washed, labeled by contacting the sample with the YOYO1-labeled nucleic acid probe and the fluorophore—labeled anchor probe, and washed again to remove any unbound probes. Following labeling, the sample was counterstained with DAPI and imaged using fluorescence microscopy. As shown in FIG. 3B, hybridization of the anchor probe to the RCP resulted in red fluorescent puncta from the covalently-linked fluorophore within the sample. This serves as a positive control to identify the locations of RCPs on the slide.

As shown in FIG. 3C, detection of green fluorescent puncta within the sample closely associated with the locations of red puncta of the anchor probes. This indicates successful hybridization of the YOYO1-labeled nucleic acid probes to the RCP. Finally, stripping the sample of all probes and rehybridizing only the anchor probe resulted in detection of clear red fluorescent puncta with no YOYO1 green signal observed, as shown in FIG. 3D. This result demonstrates that the monomethine cyanine dye maintained its non-covalent association with the nucleic acid probe throughout in situ hybridization and did not lead to background association of the dye with the sample directly.

Overall, these results demonstrated that a probe non-covalently associated with a monomethine cyanine dyes (e.g., YOYO1) can be used for in situ detection of target nucleic acids and analysis of samples. Given the high brightness of these nucleic acid probes in in situ detection as well as the low cost of monomethine cyanine dyes, use of these nucleic acid probes may help improve sensitivity of RCP detection while decreasing cost of generating probes for these assays.

### Example 2: In Situ Detection of a Target Nucleic Acid Using a Stem-Loop Nucleic Acid Probe Associated with a Monomethine Cyanine Dye

This example describes use of a stem-loop nucleic acid probe associated with a monomethine cyanine dye (MCD) for in situ detection of a target nucleic acid at a location within a biological sample.

A tissue sample is obtained and cryosectioned onto a glass slide for processing. The tissue is fixed by incubating in 3.7% paraformaldehyde (PFA). One or more washes are performed and the tissue is then permeabilized. To prepare for probe hybridization, a wash buffer is added to the tissue section. Circularizable probes targeting a gene of interest are hybridized to target nucleic acids in the tissue sample and ligated to form circular templates for rolling circle amplification (RCA). The circularizable probes include a first end sequence and a second end sequence, each complementary to two adjacent sequences in the pseudogene. The circularizable probes also include a first detection sequence and a second detection sequence. The tissue sample is then incubated with an RCA mixture containing a strand-displacing DNA polymerase and dNTP for RCA of the circularized probes to form an RCA product (“RCP”).

Next, an MCD-stained probe specific for the first detection sequence is generated. A DNA including a hairpin region with a double-stranded stem region 35-45 nt in length and a loop region 4-8 nt in length, and a single stranded overhang 10-20 nt in length extending from one end of the stem (lml of 200 μM in a standard hybridization buffer) is incubated with 1 μL of 1 mM YOYO1 dye in DMSO for 1 hour at room temperature. To prepare an anchor probe, an anchor nucleic acid molecule is covalently linked to a fluorophore through standard methods. The anchor probe is complementary to the second detection sequence of the RCP.

The tissue sample is then washed, labeled by contacting the sample with the YOYO1-labeled nucleic acid probe and the fluorophore-labeled anchor probe, and washed again to remove any unbound probes. Following labeling, the tissue sample is counterstained with DAPI and imaged using fluorescence microscopy. Hybridization of the anchor probe to the RCP will result in red fluorescent puncta from the covalently-linked fluorophore within the tissue sample. This serves as a positive control to identify the locations of RCPs on the slide.

Detection of green fluorescent puncta within the tissue sample will closely associate with the red puncta of the anchor probes, to indicate successful hybridization of the YOYO1-labeled nucleic acid probes to the RCP. Stripping the tissue sample of all probes and rehybridizing only the anchor probe will result in detection of clear red fluorescent puncta with no YOYO1 green signal observed. This example will demonstrate that the monomethine cyanine dye maintained its non-covalent association with the stem-loop nucleic acid probe throughout in situ hybridization without background association of the dye with the tissue sample directly.

Overall, these examples will demonstrate that monomethine cyanine dyes (e.g., YOYO1) are capable of non-covalently associating with stem-loop nucleic acids for use in in situ detection of target nucleic acids and analysis of tissue samples. Given the high brightness of these nucleic acid probes in in situ detection as well as the low cost of monomethine cyanine dyes, use of these nucleic acid probes may help improve sensitivity of RCP detection while decreasing the cost of generating probes for these assays.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method, comprising:
(a) contacting a biological sample comprising a target nucleic acid or an extension or amplification product thereof with a nucleic acid probe and a monomethine cyanine dye or a salt thereof,
wherein the nucleic acid probe is non-covalently associated with the monomethine cyanine dye or the salt thereof,
thereby hybridizing the nucleic acid probe to the target nucleic acid or the extension or amplification product thereof at a location in the biological sample; and
(b) detecting, at the location, a signal associated with the monomethine cyanine dye or the salt thereof.

2. The method of claim 1, wherein the monomethine cyanine dye or the salt thereof is selected from the group consisting of a thioalkyl derivative, 2-iminobenzo-thiazoline, a sulfobetaine salt of N-alkyl heterocycle, 2-chloroquinoline, 4-chloroquinoline, 3-phenyl-2H-1,4-benzothiazine, indole-3-carboxaldehyde, methyl-pyridinium iodide, thiazole orange or a derivative thereof, oxazole yellow or a derivative thereof, and oxazole blue or a derivative thereof.

3. The method of claim 1, wherein the nucleic acid probe is in a single-stranded form prior to the hybridizing.

4. The method of claim 1, wherein the monomethine cyanine dye is a monomeric monomethine cyanine dye.

5. The method of claim 4, wherein the monomeric monomethine cyanine dye has the structure: 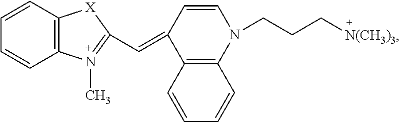
wherein X is O or S.

6. The method of claim 4, wherein the monomeric monomethine cyanine dye has the structure: 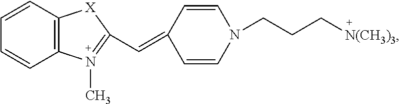
wherein X is O or S.

7. The method of claim 4, wherein the monomeric monomethine cyanine dye has the structure:

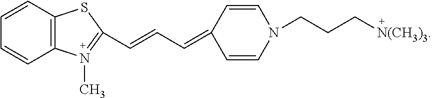

8. The method of claim 1, wherein the nucleic acid probe is in a partially double-stranded form prior to the hybridizing.

9. The method of claim 8, wherein the partially double-stranded form comprises a hairpin form.

10. The method of claim 1, wherein the monomethine cyanine dye is a dimeric monomethine cyanine dye.

11. The method of claim 10, wherein the dimeric monomethine cyanine dye has the structure: 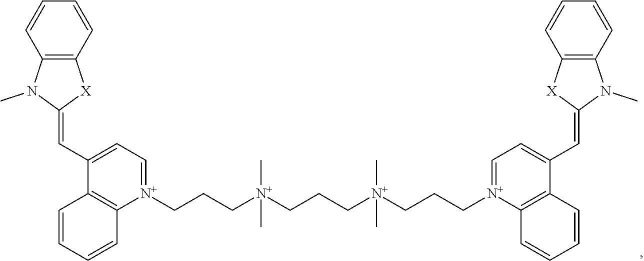
wherein X is S or O.

12. The method of claim 10, wherein the dimeric monomethine cyanine dye has the structure: 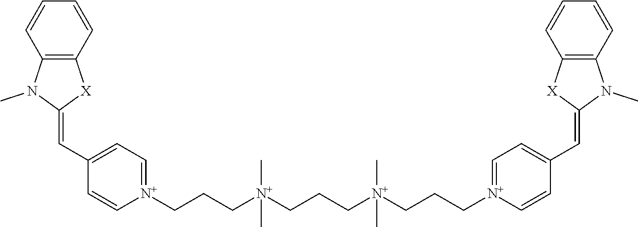
wherein X is S or O.

13. The method of claim 10, wherein the dimeric monomethine cyanine dye has the structure:

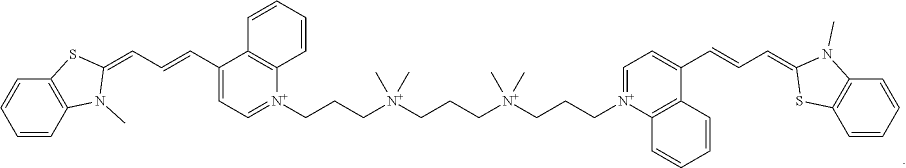 [SMILES: CN1/C(=C/C=C/c2cc[n+](CCC[N+](C)(C)CCC[N+](C)(C)CCC[n+]3ccc(/C=C/C=C4/Sc5ccccc5N4C)c4ccccc43)c3ccccc23)Sc2ccccc21]

14. The method of claim 1, further comprising, prior to the contacting, incubating the nucleic acid probe in a solution comprising the monomethine cyanine dye or the salt thereof, thereby non-covalently associating the nucleic acid probe with the monomethine cyanine dye or the salt thereof.

15. The method of claim 1, wherein the contacting is for a duration of about 30 seconds to about 2 minutes.

16. The method of claim 1, wherein the contacting comprises hybridizing the nucleic acid probe to a rolling circle amplification product (RCP) associated with a target nucleic acid at the location in the biological sample.

17. The method of claim 16, wherein the RCP is generated from a circular template at the location in the biological sample.

18. The method of claim 17, further comprising contacting the biological sample with a circularizable probe or probe set that directly or indirectly binds to the target nucleic acid at the location in the biological sample, and circularizing the circularizable probe or probe set to generate the circular template.

19. The method of claim 1, wherein the detecting comprises detecting a fluorescence signal of the monomethine cyanine dye or the salt thereof using fluorescence-based microscopy.

20. A method, comprising:
(a) contacting a biological sample comprising an amplicon of a target nucleic acid molecule with (i) a nucleic acid probe comprising a single stranded region and a double-stranded region, and (ii) a double-intercalating monomethine cyanine dye or a salt thereof that is non-covalently associated with the double-stranded region,
thereby hybridizing the nucleic acid probe to the amplicon of the target nucleic acid molecule at a location in the biological sample; and
(b) detecting, at the location, a signal associated with the monomethine cyanine dye or the salt thereof.

21. A system for detecting a target nucleic acid, the system comprising:
(a) a biological sample comprising a target nucleic acid or an extension or amplification product thereof, and
(b) a nucleic acid probe non-covalently associated with a monomethine cyanine dye or a salt thereof, wherein the nucleic acid probe is hybridized to the target nucleic acid or the extension or amplification product thereof.

22. A kit comprising:
(a) a circularizable probe or probe set that is complementary to a target nucleic acid of a biological sample; and
(b) a nucleic acid probe non-covalently associated with a monomethine cyanine dye or a salt thereof, wherein the nucleic acid probe comprises a sequence complementary to an extension or amplification product of the target nucleic acid.
